SEQUENCE LISTING (9 sequences)

SEQ ID NO: 1 - DNA, 20 bp, Artificial Sequence
  Description of Artificial Sequence Synthetic probe
cgttactcggatgcccaaat

SEQ ID NO: 2 - DNA, 20 bp, Artificial Sequence
  Description of Artificial Sequence Synthetic probe
atctttgacaactaacttac

SEQ ID NO: 3 - DNA, 20 bp, Artificial Sequence
  Description of Artificial Sequence Synthetic probe
cttgacccttacgggccatt

SEQ ID NO: 4 - DNA, 20 bp, Artificial Sequence
  Description of Artificial Sequence Synthetic probe
ttctcatcgctctacggact

SEQ ID NO: 5 - DNA, 18 bp, Artificial Sequence
  Description of Artificial Sequence Synthetic probe
atatacacccaggctccc

SEQ ID NO: 6 - DNA, 20 bp, Artificial Sequence
  Description of Artificial Sequence Synthetic probe
gcctaaccgctcagtgataa

SEQ ID NO: 7 - DNA, 20 bp, Artificial Sequence
  Description of Artificial Sequence Synthetic probe
tacgacaacaactacttaaa

SEQ ID NO: 8 - DNA, 20 bp, Artificial Sequence
  Description of Artificial Sequence Synthetic probe
agcatcttggtgcgtatccc

SEQ ID NO: 9 - DNA, 20 bp, Artificial Sequence
  Description of Artificial Sequence Synthetic probe
tgcatttgccgtcaactgga

(19) United States
(12) Patent Application Publication
(10) Pub. No.: US 2014/0072962 A1
(43) Pub. Date: Mar. 13, 2014

(54) SYSTEMS, DEVICES, AND METHODS FOR IDENTIFYING A DISEASE STATE IN A BIOLOGICAL HOST USING INTERNAL CONTROLS

(71) Applicant: Xagenic Inc., Toronto (CA)

(72) Inventors: Shana O. Kelley, Toronto (CA); Graham Jack, Toronto (CA)

(73) Assignee: Xagenic Inc., Toronto (CA)

(21) Appl. No.: 13/963,656

(22) Filed: Aug. 9, 2013

Related U.S. Application Data
(60) Provisional application No. 61/700,285, filed on Sep. 12, 2012.

(51) Int. Cl. C12Q 1/70; C12Q 1/68

(52) U.S. Cl. CPC C12Q 1/701; C12Q 1/6888
USPC 435/5; 435/287.2; 435/6.15

(57) ABSTRACT

Contemplated methods and devices comprise detecting the presence of a pathogen in a biological host. In certain implementations, a sample is provided from a biological host. A biosensor is provided, the biosensor having a first probe configured to detect a control marker in the sample, the control marker being an endogenous element of the biological host. The biosensor has a second probe configured to detect the presence of a target marker in the sample, the target marker being from a pathogen in the biological host. The sample is applied to the biosensor, and the presence or absence of the control marker in the sample is identified using the first probe. The presence or absence of the target marker in the sample is identified using the second probe.

### CROSS-REFERENCE TO RELATED APPLICATION

This application claims the benefit of priority to U.S. Provisional Patent Application No. 61/700,285, filed Sep. 12, 2012, which is hereby incorporated by reference herein in its entirety.

### FIELD OF THE INVENTION

The field of the invention is analytical devices for characterizing or detecting a wide range of analytes, including nucleic acids, proteins and small molecules.

### BACKGROUND

Diagnostic tests for various diseases can provide important information for successful treatment. Diagnostic assays are used to detect pathogens, including bacteria and viruses. Many standard diagnostic assays, such as cell cultures and genetic testing with PCR amplification, require sending samples to labs and have long turnaround times of several days or weeks. Many patients, in such cases, do not return to the care provider to receive the results or treatments, and in some cases, the long turnaround can compromise the ability to properly treat the condition.

While some assays have been automated, many still require significant expertise or training. For example, lab technicians commonly process biological samples through assays, but typically rely on multiple external test controls to ensure that the assay was performed correctly.

Alternative systems and methods for diagnostics could be beneficial for improved patient outcomes, particularly in point-of-care applications.

### SUMMARY

Disclosed herein are systems, devices, and methods for detecting the presence of a pathogen in a biological host, such as in a point-of-care setting. In certain aspects, a method includes providing a sample from a biological host, and applying the sample to a biosensor that utilizes an internal control. In certain embodiments, the biosensor includes a first probe configured to detect a control marker in the sample, the control marker being an endogenous element of the biological host, and a second probe configured to detect the presence of a target marker in the sample, the target marker being from a pathogen in the biological host. The first probe is used to identify the presence or absence of the control marker in the sample and the second probe is used to identify the presence or absence of the target marker in the sample.

In certain embodiments, the biosensor for detecting the presence of a pathogen includes a first sensor and a second sensor, with a first probe coupled to the first sensor and a second probe coupled to the second sensor. In certain approaches, the biosensor includes a third probe. For example, the third probe may be a non-sense probe comprising a peptide nucleic acid. In certain approaches, the third probe is coupled to a third sensor of the biosensor. The method may include the step of applying a lysing procedure to the sample before applying the sample to the biosensor. In certain approaches, the lysing procedure is an electrochemical lysing procedure. In certain embodiments, identifying the presence or absence of a marker in the sample comprises measuring an electrocatalytic signal at the biosensor. In certain approaches, at least one of the control marker and the target marker comprises a ribonucleic acid sequence. In certain approaches, at least one of the first and second probes comprises a peptide nucleic acid sequence tethered to the biosensor with a thiol bond.

Methods for detecting the presence of a pathogen in a biological host may include the steps of receiving a first biosensor signal indicative of the presence of the control marker in the sample, receiving a second biosensor signal indicative of the presence of the target marker in the sample, and determining, based on the first biosensor signal and the second biosensor signal, that the pathogen is present in the biological host. Additionally or alternatively, methods may include the steps of receiving a first biosensor signal indicative of the presence of the control marker in the sample, receiving a second biosensor signal indicative of the absence of the target marker in the sample, and determining, based on the first biosensor signal and the second biosensor signal, that the pathogen is not present in the biological host. In certain approaches, methods include receiving a first biosensor signal indicative of the absence of the control marker in the sample, and determining, based on the first signal, an error.

In certain embodiments, methods for detecting the presence of a pathogen in a biological host may include the step of performing a baseline measurement using the first probe before applying the sample to the biosensor. Identifying the presence of the control marker may be performed by comparing the baseline measurement from the first probe with a measurement performed using the first probe after applying the sample to the biosensor. In certain approaches, the method comprises performing a baseline measurement using the second probe before applying the sample to the biosensor. Identifying the presence of the target marker may be performed by comparing the baseline measurement from the second probe with a measurement performed using the second probe after applying the sample to the biosensor.

In certain approaches, methods of detection include applying an electrocatalytic reagent to the biosensor. For example, a redox pair having a first transition metal complex and a second transition metal complex may be added to the sample to amplify the electrocatalytic signal. In certain approaches, identifying the presence of the control marker includes applying a voltage signal to the first probe, and measuring a current signal from an electrode. In certain approaches, identifying the presence of the target marker comprises applying a voltage signal to the second probe, and measuring a current signal from an electrode.

A first probe is provided to detect a control marker. The first probe may comprise at least one of nucleic acids, peptide nucleic acids, locked nucleic acids, proteins, or peptides functionalized with suitable tethering molecules. In certain approaches, the first probe is a nucleic acid sequence tethered to a first location on the biosensor. In certain approaches, the first probe is a peptide nucleic acid probe tethered to a first location on the biosensor. In certain approaches, the first probe is tethered to the biosensor with a thiol bond. The control marker comprises at least one of nucleic acids, proteins, or peptides. In certain embodiments, the control marker comprises a nucleic acid sequence. In certain approaches, the control marker is from a human epithelial cell.

A second probe is provided to detect a target marker. The second probe may comprise at least one of nucleic acids, peptide nucleic acids, locked nucleic acids, proteins, or peptides functionalized with suitable tethering molecules. In certain approaches, the second probe is a nucleic acid sequence tethered to a second location on the biosensor. In certain approaches, the second probe is a peptide nucleic acid probe tethered to a second location on the biosensor. In certain approaches, the second probe is tethered to the biosensor with a thiol bond. The target marker comprises at least one of nucleic acids, proteins, or peptides. In certain embodiments, the target marker comprises a nucleic acid sequence. In certain approaches, the target marker is from a pathogen, and the pathogen is bacteria. In certain approaches, the bacteria is Chlamydia trachomatis.

In certain approaches, methods of detection include contacting the sample to the first probe and the second probe under hybridization conditions. In certain approaches, the first probe and second probe are located in a chamber. Applying the sample to the biosensor may include flowing the sample through the chamber at a flow rate. For example, the flow rate may be fixed or variable. In certain approaches, the flow is laminar. In certain approaches, applying the sample to the biosensor comprises agitating the sample.

In certain approaches, a first sensor is provided. For example, the first probe may be coupled to the first sensor. In certain approaches, the first sensor is conductive. In certain embodiments, the first sensor is a microelectrode. The first sensor may be a nanostructured microelectrode. In certain approaches, the first sensor comprises at least one of gold, platinum, and palladium. The first sensor may include a plurality of sensors.

In certain approaches, a second sensor is provided. For example, the second probe may be coupled to the second sensor. In certain approaches, the second sensor is conductive. In certain embodiments, the second sensor is a microelectrode. The second sensor may be a nanostructured microelectrode. In certain approaches, the second sensor comprises at least one of gold, platinum, and palladium. The second sensor may include a plurality of sensors.

In certain embodiments, the biosensor includes a third sensor. For example, the third sensor may have a third probe. In certain approaches, the third probe is a non-sense probe. The third probe may comprise at least one of nucleic acids, peptide nucleic acids, locked nucleic acids, proteins, or peptides functionalized with suitable tethering molecules. In certain approaches, the third probe is a nucleic acid sequence tethered to a third location on the biosensor. In certain approaches, the third probe is a peptide nucleic acid probe tethered to a third location on the biosensor. In certain approaches, the first probe is tethered to the biosensor with a thiol bond.

In certain approaches, a third sensor is provided. For example, the third probe may be coupled to the third sensor. In certain approaches, the third sensor is conductive. In certain embodiments, the third sensor is a microelectrode. The third sensor may be a nanostructured microelectrode. In certain approaches, the third sensor comprises at least one of gold, platinum, and palladium. The third sensor may include a plurality of sensors.

In certain aspects, a method for detecting the presence of a pathogen in a biological host includes providing a sample from a biological host, providing a biosensor having a first conductive sensor with a first nucleic acid probe configured to detect the presence of a control marker in the sample and a second conductive sensor with a second nucleic acid probe configured to detect the presence of a target marker in the sample, applying the sample to a biosensor, applying an electrocatalytic reagent to the biosensor, measuring a first electrocatalytic signal at the first conductive sensor generated by hybridization of the control marker with the first nucleic acid probe, and measuring a second electrocatalytic signal at the conductive second sensor generated by hybridization of the target marker with the second nucleic acid probe. In certain approaches, the control marker is an endogenous element of the biological host, and the target marker is from a pathogen in the biological host.

In certain approaches, the methods provide the step of applying a lysing procedure to the sample before applying the sample to the biosensor. In certain approaches, the lysing procedure is an electrochemical lysing procedure. In certain embodiments, methods for detecting the presence of a pathogen in a biological host may include the step of performing a baseline measurement at the first conductive sensor before applying the sample to the biosensor. Identifying the presence of the control marker may be performed by comparing the baseline measurement of the first conductive sensor with the first electrocatalytic signal. In certain approaches, the method comprises performing a baseline measurement at the second conductive sensor before applying the sample to the biosensor. Identifying the presence of the target marker may be performed by comparing the baseline measurement of second conductive sensor with the second electrocatalytic signal.

In certain approaches, methods of detection include applying an electrocatalytic reagent to the biosensor. For example, a redox pair having a first transition metal complex and a second transition metal complex may be added to the sample to amplify the electrocatalytic signal. In certain approaches, measuring the first electrocatalytic signal includes applying a voltage signal to the first sensor, and measuring a current signal from an electrode. In certain approaches, measuring the first electrocatalytic signal includes applying a voltage signal to the second sensor, and measuring a current signal from an electrode.

A first probe is provided to detect a control marker. The first probe may comprise at least one of nucleic acids, peptide nucleic acids, locked nucleic acids, proteins, or peptides functionalized with suitable tethering molecules. In certain approaches, the first probe is a nucleic acid sequence tethered to a first sensor. In certain approaches, the first probe is a peptide nucleic acid probe tethered to a first sensor. In certain approaches, the first probe is tethered to the biosensor with a thiol bond. The control marker comprises at least one of nucleic acids, proteins, or peptides. In certain embodiments, the control marker comprises a nucleic acid sequence. In certain embodiments, the control marker comprises a ribonucleic acid sequence. In certain approaches, the control marker is from a human epithelial cell.

A second probe is provided to detect a target marker. The second probe may comprise at least one of nucleic acids, peptide nucleic acids, locked nucleic acids, proteins, or peptides functionalized with suitable tethering molecules. In certain approaches, the second probe is a nucleic acid sequence tethered to a second sensor. In certain approaches, the second probe is a peptide nucleic acid probe tethered to a second sensor. In certain approaches, the second probe is tethered to the biosensor with a thiol bond. The target marker comprises at least one of nucleic acids, proteins, or peptides. In certain embodiments, the target marker comprises a nucleic acid sequence. In certain embodiments, the target marker comprises a ribonucleic acid sequence. In certain approaches, the target marker is from a pathogen, and the pathogen is bacteria. In certain approaches, the bacteria is Chlamydia trachomatis.

In certain approaches, methods of detection include contacting the sample to the first conductive sensor and the second conductive sensor under hybridization conditions. In certain approaches, the first conductive sensor and second conductive sensor are located in a chamber. Applying the sample to the biosensor may include flowing the sample through the chamber at a flow rate. For example, the flow rate may be fixed or variable. In certain approaches, the flow is laminar. In certain approaches, applying the sample to the biosensor comprises agitating the sample.

In certain embodiments, the first conductive sensor is a microelectrode. The first conductive sensor may be a nanostructured microelectrode. In certain approaches, the first conductive sensor comprises at least one of gold, platinum, and palladium. The first conductive sensor may include a plurality of sensors. In certain embodiments, the second conductive sensor is a microelectrode. The second conductive sensor may be a nanostructured microelectrode. In certain approaches, the second conductive sensor comprises at least one of gold, platinum, and palladium. The second conductive sensor may include a plurality of sensors.

In certain embodiments, the biosensor includes a third conductive sensor. For example, the third conductive sensor may have a third probe. In certain approaches, the third probe is a non-sense probe. The third probe may comprise at least one of nucleic acids, peptide nucleic acids, locked nucleic acids, proteins, or peptides functionalized with suitable tethering molecules. In certain approaches, the third probe is a nucleic acid sequence tethered to the third conductive sensor. In certain approaches, the third probe is a peptide nucleic acid probe tethered to the third conductive sensor. In certain approaches, the first probe is tethered to the third conductive sensor with a thiol bond. In certain embodiments, a third conductive sensor is a microelectrode. The third conductive sensor may be a nanostructured microelectrode. The third conductive sensor may comprise at least one of gold, platinum, and palladium. The third conductive sensor may include a plurality of sensors.

In certain aspects of the systems and devices described herein, a biosensor is provided for detecting the presence of a pathogen in a biological host. The biosensor includes a solid support base with a sensor affixed to the support base. The sensor includes a first probe configured to detect the presence of a control marker. In certain approaches, the control marker is an endogenous element of a biological host. The sensor includes a second probe configured to detect the presence of a target marker. In certain approaches, the target marker is from a pathogen in the biological host.

In certain embodiments, the first probe comprises a nucleic acid sequence tethered to the sensor. In certain embodiments, the second probe comprises a peptide nucleic acid sequence tethered to the sensor. In some approaches, at least one of the first and second probe is tethered to the sensor with a thiol bond. In some embodiments, at least one of the control marker and target marker comprises a ribonucleic acid sequence. The biosensor may include a third probe. For example, the third probe may be a non-sense probe comprising a peptide nucleic acid.

A first probe is provided to detect a control marker. The first probe may comprise at least one of nucleic acids, peptide nucleic acids, locked nucleic acids, proteins, or peptides functionalized with suitable tethering molecules. In certain approaches, the first probe is a nucleic acid sequence tethered to a first location on the biosensor. In certain approaches, the first probe is a peptide nucleic acid probe tethered to a first location on the biosensor. In certain approaches, the first probe is tethered to the biosensor with a thiol bond. The control marker comprises at least one of nucleic acids, proteins, or peptides. In certain embodiments, the control marker comprises a nucleic acid sequence. In certain embodiments, the control marker comprises a ribonucleic acid sequence. In certain approaches, the control marker is from a human epithelial cell.

A second probe is provided to detect a target marker. The second probe may comprise at least one of nucleic acids, peptide nucleic acids, locked nucleic acids, proteins, or peptides functionalized with suitable tethering molecules. In certain approaches, the second probe is a nucleic acid sequence tethered to a second location on the biosensor. In certain approaches, the second probe is a peptide nucleic acid probe tethered to a second location on the biosensor. In certain approaches, the second probe is tethered to the biosensor with a thiol bond. The target marker comprises at least one of nucleic acids, proteins, or peptides. In certain embodiments, the target marker comprises a nucleic acid sequence. In certain embodiments, the target marker comprises a ribonucleic acid sequence. In certain approaches, the target marker is from a pathogen, and the pathogen is bacteria. In certain approaches, the bacteria is Chlamydia trachomatis.

In certain approaches, the biosensor includes a solid support base. In certain approaches, the solid support base comprises a printed circuit board. Additionally or alternatively, the solid support base may comprise silicon. In certain approaches, the first sensor and second sensor are located in a chamber of the biosensor. The first sensor and second sensor may be located linearly along a length of the chamber.

In certain approaches, the first sensor is conductive. In certain embodiments, the first sensor is a microelectrode. The first sensor may be a nanostructured microelectrode. In certain approaches, the first sensor comprises at least one of gold, platinum, and palladium. The first sensor may include a plurality of sensors. In certain approaches, the second sensor is conductive. In certain embodiments, the second sensor is a microelectrode. The second sensor may be a nanostructured microelectrode. In certain approaches, the second sensor comprises at least one of gold, platinum, and palladium. The second sensor may include a plurality of sensors.

In certain embodiments, the biosensor includes a third sensor. For example, the third sensor may have a third probe. In certain approaches, the third probe is a non-sense probe. The third probe may comprise at least one of nucleic acids, peptide nucleic acids, locked nucleic acids, proteins, or peptides functionalized with suitable tethering molecules. In certain approaches, the third probe is a nucleic acid sequence tethered to a third location on the biosensor. In certain approaches, the third probe is a peptide nucleic acid probe tethered to a third location on the biosensor. In certain approaches, the first probe is tethered to the biosensor with a thiol bond. In certain approaches, a third sensor is conductive. In certain embodiments, the third sensor is a microelectrode. The third sensor may be a nanostructured microelectrode. In certain approaches, the third sensor comprises at least one of gold, platinum, and palladium. The third sensor may include a plurality of sensors.

In certain embodiments, the biosensor comprises an inlet channel having a first width coupled to a first end of the chamber, and the chamber has a second width. The first width and second width may be approximately equal. In certain approaches, the biosensor includes a lysing chamber. In certain embodiments, the lysing chamber includes at least one electrode. The electrode of the lysing chamber may include at least one of copper, nickel, and gold.

In certain aspects, a biosensor is provided having an inlet channel, a chamber having a first end coupled to the inlet channel, an outlet channel coupled to a second end of the chamber, a base coupled to the chamber and forming a support along a length of the chamber, a first sensor affixed to the base and positioned within the chamber, a second sensor affixed to the base and positioned within the chamber. The second sensor is aligned with the first sensor along the length of the chamber. The first sensor includes a first probe configured to detect the presence of a control marker, the control marker being an endogenous element of a biological host. The second sensor includes a second probe configured to detect the presence of a target marker, the target marker being from a pathogen from the biological host.

In certain embodiments, the inlet channel has a first width and the chamber has a second width, and the first width and second width are approximately equal. In certain approaches, the outlet channel has a third width, wherein the third width is approximately the equal to the first width of the inlet channel.

A first probe is provided to detect a control marker. The first probe may comprise at least one of nucleic acids, peptide nucleic acids, locked nucleic acids, proteins, or peptides functionalized with suitable tethering molecules. In certain approaches, the first probe is a nucleic acid sequence tethered to the first sensor. In certain approaches, the first probe is a peptide nucleic acid probe tethered to the first sensor. In certain approaches, the first probe is tethered to the first sensor with a thiol bond. The control marker comprises at least one of nucleic acids, proteins, or peptides. In certain embodiments, the control marker comprises a nucleic acid sequence. In certain embodiments, the control marker comprises a ribonucleic acid sequence. In certain approaches, the control marker is from a human epithelial cell.

A second probe is provided to detect a target marker. The second probe may comprise at least one of nucleic acids, peptide nucleic acids, locked nucleic acids, proteins, or peptides functionalized with suitable tethering molecules. In certain approaches, the second probe is a nucleic acid sequence tethered to the second sensor. In certain approaches, the second probe is a peptide nucleic acid probe tethered to the second sensor. In certain approaches, the second probe is tethered to the second sensor with a thiol bond. The target marker comprises at least one of nucleic acids, proteins, or peptides. In certain embodiments, the target marker comprises a nucleic acid sequence. In certain embodiments, the target marker comprises a ribonucleic acid sequence. In certain approaches, the target marker is from a pathogen, and the pathogen is bacteria. In certain approaches, the bacteria is Chlamydia trachomatis.

In certain approaches, the biosensor includes a solid support base. In certain approaches, the solid support base comprises a printed circuit board. Additionally or alternatively, the solid support base may comprise silicon. In certain approaches, the first sensor and second sensor are located in a chamber of the biosensor. The first sensor and second sensor may be located linearly along a length of the chamber.

In certain approaches, the first sensor is conductive. In certain embodiments, the first sensor is a microelectrode. The first sensor may be a nanostructured microelectrode. In certain approaches, the first sensor comprises at least one of gold, platinum, and palladium. The first sensor may include a plurality of sensors. In certain approaches, the second sensor is conductive. In certain embodiments, the second sensor is a microelectrode. The second sensor may be a nanostructured microelectrode. In certain approaches, the second sensor comprises at least one of gold, platinum, and palladium. The second sensor may include a plurality of sensors.

In certain embodiments, the biosensor includes a third sensor. For example, the third sensor may have a third probe. In certain approaches, the third probe is a non-sense probe. The third probe may comprise at least one of nucleic acids, peptide nucleic acids, locked nucleic acids, proteins, or peptides functionalized with suitable tethering molecules. In certain approaches, the third probe is a nucleic acid sequence tethered to a third location on the biosensor. In certain approaches, the third probe is a peptide nucleic acid probe tethered to a third location on the biosensor. In certain approaches, the first probe is tethered to the biosensor with a thiol bond. In certain approaches, a third sensor is conductive. In certain embodiments, the third sensor is a microelectrode. The third sensor may be a nanostructured microelectrode. In certain approaches, the third sensor comprises at least one of gold, platinum, and palladium. The third sensor may include a plurality of sensors.

In certain approaches, the biosensor includes a lysing chamber. In certain embodiments, the lysing chamber includes at least one electrode. The electrode of the lysing chamber may include at least one of copper, nickel, and gold.

In certain approaches, methods for detecting the presence of a pathogen include receiving a signal indicative of the presence of the control marker. The signal indicative of the presence of the control marker may be indicative of a quantity of the endogenous element of the biological host. The signal indicative of the presence of the control marker may be indicative of components obtained from a lysing procedure. The signal indicative of the presence of the control marker may be indicative of hybridization between a sequence of the first probe and a sequence of the control marker. In certain approaches, methods for detecting the presence of a pathogen include receiving a first signal indicative of the absence of the control marker, and signalling, based on the first indication, an error. The error may be one of receiving an insufficient quantity of matter from the biological host, improperly performing a lysis procedure on the biological sample, and providing inadequate hybridization conditions.

In certain approaches, methods for detecting the presence of a pathogen include receiving a signal indicative of the presence of the target marker in the biological sample. The signal indicative of the presence of the target marker is indicative of a quantity of the target marker from the pathogen. In certain approaches, methods include determining, based on the first biosensor signal indicative of the presence of the control marker and the second biosensor signal indicative of the presence of the target marker, that the pathogen is present in the biological host.

In certain embodiments, methods include receiving a second biosensor signal indicative of the absence of the target marker. In certain approaches, the second biosensor signal is indicative of an absence of a quantity of the target marker from the pathogen. The method may include determining, based on the first biosensor signal indicative of the presence of the control marker and the second biosensor signal indicative of the absence of the target marker, that the pathogen is not present in the biological host.

In certain approaches, methods for detecting the presence of a pathogen include receiving a third biosensor signal indicative of hybridization at the non-sense probe. The method may further include signalling an error based on the third biosensor signal. In certain embodiments, the method includes determining that the pathogen is present in the biological host based on a first biosensor signal indicative of the presence of the control marker, a second biosensor signal indicative of the presence of the target marker, and a third biosensor signal indicative of an absence of hybridization at the non-sense probe. In certain embodiments, the method includes determining that the pathogen is not present in the biological host based on a first biosensor signal indicative of the presence of the control marker, a second biosensor signal indicative of the absence of the target marker, and a third biosensor signal indicative of an absence of hybridization at the non-sense probe.

In certain approaches, methods for detecting the presence of a pathogen include applying a lysing procedure to the sample before applying the sample to the biosensor. In certain approaches, the lysing procedure is an electrochemical lysing procedure.

In certain approaches, a first biosensor signal is received from the first probe. For example, the first biosensor signal may be received from a first sensor. In certain approaches, the first probe is coupled to a first sensor. In certain approaches, a second biosensor signal is received from the second probe. For example, the second biosensor signal may be received from a second sensor. In certain approaches, the second probe is coupled to a second sensor. In certain approaches, a third biosensor signal is received from a third probe. For example, the third biosensor signal may be received from a third sensor. In certain approaches, the third probe is coupled to a third sensor.

In certain approaches, the devices and systems described herein include a first indicator configured to indicate the presence or absence of the control marker at the first sensor. The devices and systems described herein may include a second indicator configured to indicate the presence or absence of the target marker at the first sensor. The devices and systems described herein may include a third indicator configured to indicate the presence or absence of hybridization at the third sensor.

In certain approaches, methods for detecting the presence of a pathogen include identifying the presence or absence of the control marker by measuring an electrocatalytic signal at a first sensor. Identifying the presence or absence of the target marker may include measuring an electrocatalytic signal at a second sensor. In certain approaches, the first probe comprises a peptide nucleic acid tethered to a first sensor with a thiol bond. In certain approaches, the second probe comprises a peptide nucleic acid probe tethered to a second sensor with a thiol bond. In certain approaches, a baseline measurement is an electrocatalytic measurement. In certain approaches, an electrocatalytic reagent is a redox pairing having a first transition metal complex and a second transition metal complex.

Variations and modifications of these embodiments will occur to those of skill in the art after reviewing this disclosure. The foregoing features and aspects may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 depicts electrocatalytic detection of a nucleotide strand;

FIG. 2 depicts electrocatalytic detection signals;

FIGS. 3A-3D depict a nanostructured microelectrode system for electrocatalytic detection of a nucleotide strand;

FIGS. 4-9 depict analysis chambers;

FIGS. 10A-10B depict flow of a sample through an analysis chamber;

FIG. 11 depicts an embodiment of an electrode configuration for an analysis chamber;

FIG. 12 depicts an electrical lysis chamber;

FIG. 13 depicts a system for preparing and analyzing a biological sample;

FIG. 14 depicts an interpretation table for application of a two-probe system;

FIG. 15 depicts an interpretation table for application of a three-probe system;

FIG. 16 depicts a cartridge system for receiving, preparing, and analyzing a biological sample;

FIG. 17 depicts a cartridge for an analytical detection system; and

FIG. 18 depicts an automated testing system.

### DETAILED DESCRIPTION

To provide an overall understanding of the systems, devices, and methods described herein, certain illustrative embodiments will be described. It is to be understood that the systems, devices, and methods disclosed herein, while shown for use in diagnostic systems for bacterial diseases such as Chlamydia, may be applied in other applications including, but not limited to, detection of other bacteria, viruses, fungi, prions, plant matter, animal matter, protein, RNA sequences, DNA sequences, as well as cancer screening and genetic testing, including screening for genetic traits and disorders.

Disclosed herein are systems, devices, and methods for detecting the presence of a pathogen in a biological host, such as in a point-of-care setting. In certain aspects, a method includes providing a sample from a biological host, and applying the sample to a biosensor that utilizes an internal control. In certain application, the biosensor includes a first probe configured to detect a control marker in the sample, the control marker being an endogenous element of the biological host, and a second probe configured to detect the presence of a target marker in the sample, the target marker being from a pathogen in the biological host. The first probe is used to identify the presence or absence of the control marker in the sample and the second probe is used to identify the presence or absence of the target marker in the sample.

The systems, devices, and methods described herein may be used for diagnosing a disease in a living organism, such as a human or animal. For example, Chlamydia is a bacterial disease that afflicts humans and is caused by the bacteria Chlamydia trachomatis. A caretaker, such as a nurse or physician, may obtain a sample from a patient desiring to receive a diagnosis for this disorder. For example, the caretaker may use a medical swab to wipe the surface of the vagina, to thereby obtain a biological sample of vaginal fluid and vaginal epithelial cells. If the patient is carrying the Chlamydia trachomatis bacteria, the bacteria would be present in the sample. Additional markers specific to the human genome would also be present. The caretaker or technician then uses the systems, devices, and methods described herein to detect the presence or absence of the bacteria or other pathogen, cell, protein, or gene in the sample.

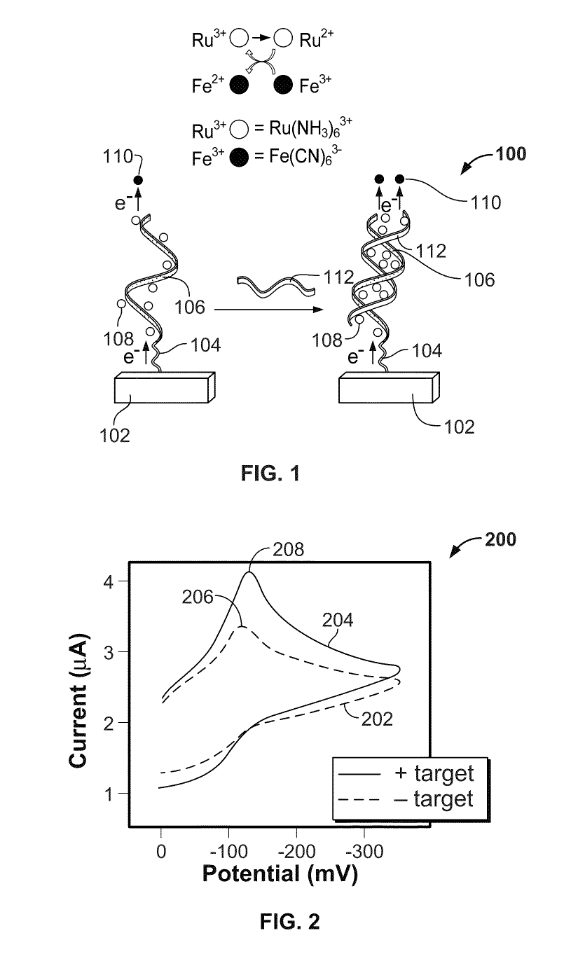

FIGS. 1-3D depict illustrative tools, sensors, biosensors, and technology for detecting cellular, molecular, or tissue components by electrocatalytic methods. Such tools and technologies are first illustrated in general followed by a discussion of various implementations and applications.

FIG. 1 depicts electrocatalytic detection of a nucleotide strand using a biosensor system. System 100 includes an electrode 102 with an associated probe 106 attached to the electrode 102 with a linker 104. The probe 106 is a molecule or group of molecules, such as nucleic acids (e.g., DNA, RNA, cDNA, mRNA, rRNA, etc.), oligonucleotides, peptide nucleic acids, locked nucleic acids, proteins (e.g., antibodies, enzymes, etc.), or peptides, that is able to bind to or otherwise interact with a biomarker target (e.g., receptor, ligand) to provide an indication of the presence of the ligand or receptor in a sample. The linker 104 is a molecule or group of molecules which tethers the probe 106 to the electrode 102, for example, through a chemical bond, such as a thiol bond.

In certain embodiments, the probe 106 is a polynucleotide capable of binding to a target nucleic acid sequence through one or more types of chemical bonds, such as complementary base pairing and hydrogen bond formation. This binding is also called hybridization or annealing. For example, the probe 106 may include naturally occurring nucleotide and nucleoside bases, such as adenine (A), guanine (G), cytosine (C), thymine (T), and uracil (U), or modified bases, such as 7-deazaguanosine and inosine. The bases in probe 106 can be joined by a phosphodiester bond (e.g., DNA and RNA molecules), or with other types of bonds. For example, the probe 106 can be a peptide nucleic acid oligomer in which the constituent bases are joined by peptide bonds rather than phosphodiester linkages. A peptide nucleic acid oligomer may contain a backbone comprised of N-(2-aminoethyl)-glycine units linked by peptide bonds. Peptide nucleic acids have a higher binding affinity and increased specificity to complementary nucleic acid oligomers, and accordingly, may be particularly beneficial in diagnostic and other sensing applications, as described herein.

In certain embodiments, the probe 106 has a sequence partially or completely complementary to a target marker 112, such as a nucleic acid sequence sought. Target marker 112 is a molecule for detection, as will be described in further detail below. In certain embodiments, probe 106 is a single-stranded oligonucleotide capable of binding to at least a portion of a target nucleic acid sought to be detected. In certain approaches, the probe 106 has regions which are not complementary to a target sequence, for example, to adjust hybridization between strands or to serve as a non-sense or negative control during an assay. The probe 106 may also contain other features, such as longitudinal spacers, double-stranded regions, single-stranded regions, poly(T) linkers, and double stranded duplexes as rigid linkers and PEG spacers. In certain approaches, electrode 102 can be configured with multiple, different probes 106 for multiple, different targets 112.

The probe 106 includes a linker 104 that facilitates binding of the probe 106 to the electrode 102. In certain approaches, the linker 104 is associated with the probe 106 and binds to the electrode 102. For example, the linker 104 may be a functional group, such as a thiol, dithiol, amine, carboxylic acid, or amino group. For example, it may be 4-mercaptobenzoic acid coupled to a 5′ end of a polynucleotide probe. In certain approaches, the linker 104 is associated with the electrode 102 and binds to the probe 106. For example, the electrode 102 may include an amine, silane, or siloxane functional group. In certain approaches, the linker 104 is independent of the electrode 102 and the probe 106. For example, linker 104 may be a molecule in solution that binds to both the electrode 102 and the probe 106.

Under appropriate conditions, the probe 106 can hybridize to a complementary target marker 112 to provide an indication of the presence of target marker 112 in a sample. In certain approaches, the sample is a biological sample from a biological host. For example, a sample may be tissue, cells, proteins, fluid, genetic material, bacterial matter or viral matter, a plant, animal, cell culture, or other organism or host. The sample may be a whole organism or a subset of its tissues, cells or component parts, and may include cellular or noncellular biological material. Fluids and tissues may include, but are not limited to, blood, plasma, serum, cerebrospinal fluid, lymph, tears, saliva, blood, mucus, lymphatic fluid, synovial fluid, cerebrospinal fluid, amniotic fluid, amniotic cord blood, urine, vaginal fluid, semen, tears, milk, and tissue sections. The sample may contain nucleic acids, such as deoxyribonucleic acids (DNA), ribonucleic acids (RNA), or copolymers of deoxyribonucleic acids and ribonucleic acids or combinations thereof. In certain approaches, the target marker 112 is a nucleic acid sequence that is known to be unique to the host, pathogen, disease, or trait, and the probe 104 provides a complementary sequence to the sequence of the target marker 112 to allow for detection of the host sequence in the sample.

In certain aspects, systems, devices and methods are provided to perform processing steps, such as purification and extraction, on the sample. Analytes or target molecules for detection, such as nucleic acids, may be sequestered inside of cells, bacteria, or viruses. The sample may be processed to separate, isolate, or otherwise make accessible, various components, tissues, cells, fractions, and molecules included in the sample. Processing steps may include, but are not limited to, purification, homogenization, lysing, and extraction steps. The processing steps may separate, isolate, or otherwise make accessible a target marker, such as the target marker 112 in or from the sample.

In certain approaches, the target marker 112 is genetic material in the form of DNA or RNA obtained from any naturally occurring prokaryotes such, pathogenic or non-pathogenic bacteria (e.g., Escherichia, Salmonella, Clostridium, Chlamydia, etc.), eukaryotes (e.g., protozoans, parasites, fungi, and yeast), viruses (e.g., Herpes viruses, HIV, influenza virus, Epstein-Barr virus, hepatitis B virus, etc.), plants, insects, and animals, including humans and cells in tissue culture. Target nucleic acids from these sources may, for example, be found in biological samples of a bodily fluid from an animal, including a human. In certain approaches, the sample is obtained from a biological host, such as a human patient, and includes non-human material or organisms, such as bacteria, viruses, other pathogens.

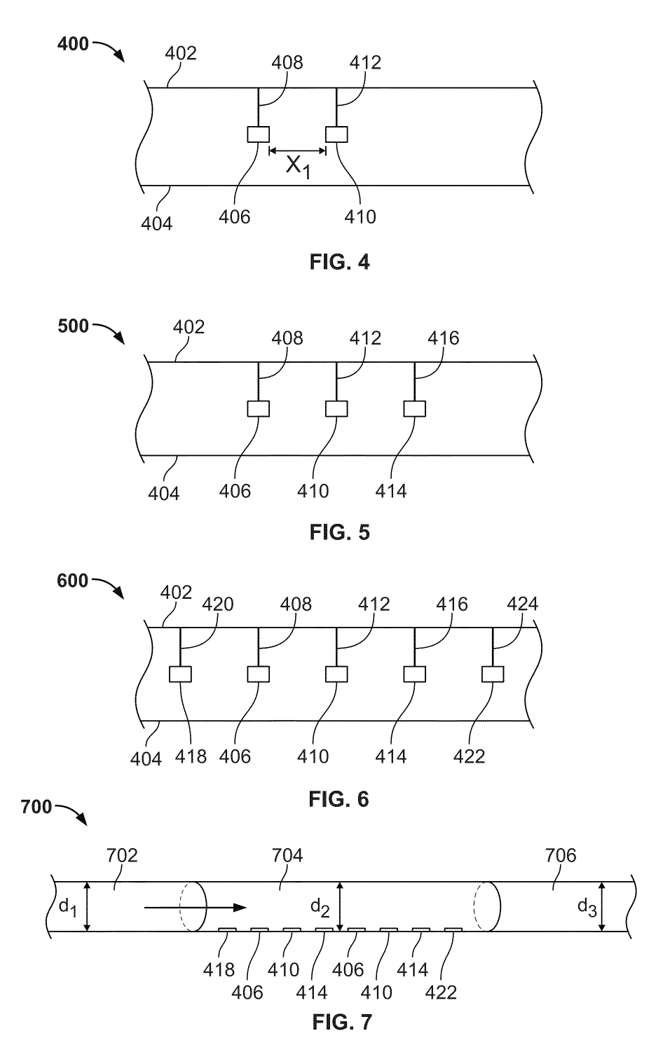

As discussed in further detail in reference to FIG. 4 et seq., a target marker from a pathogen in the patient sample can be detected by the systems, devices, and methods described herein. In certain approaches, the systems, devices, and methods described herein rely on a control marker that is also taken from the sample to detect the presence or absence of target molecules from the host and/or the bacteria and notify the caretaker of that detection in a point-of-care setting. For example, a first probe may test for a marker for an endogenous element of the biological host found in the sample (e.g., human nucleic acid sequence), for use as a control and a second probe tests for a marker for a pathogen (e.g., bacterial RNA) from that sample. As discussed further below when the endogenous element and pathogen are both detected as being present in the sample, the systems and devices can signal to the care giver and indicate that the target is detected.

A target nucleic acid molecule, such as target marker 112, may optionally be amplified prior to detection. The target nucleic acid can be in a double-stranded or single-stranded form. A double-stranded form may be treated with a denaturation agent to render the two strands into a single-stranded form, or partially single-stranded form, at the start of the amplification reaction, by methods such as heating, alkali treatment, or by enzymatic treatment.

Once the sample has been treated to expose a target nucleic acid, e.g., target molecule 112, the sample solution can be tested as described herein to detect hybridization between probe 106 and target molecule 112. For example, electrocatalytic detection may be applied as will be described in more detail below. If target molecule 112 is not present in the sample, the systems, device, and methods described herein may detect the absence of the target molecule. For example, in the case of diagnosing a bacterial pathogen, such as Chlamydia trachomatis, the presence in the sample of a target molecule, such as an RNA sequence from Chlamydia trachomatis, would indicate presence of the bacteria in the biological host (e.g., a human patient), and the absence of the target molecule in the sample indicates that the host is not infected with Chlamydia trachomatis. Similarly, other markers may be used for other pathogens and diseases.

Referring to FIG. 1, the probe 106 of the system 100 hybridizes to a complementary target molecule 112. In certain approaches, the hybridization is through complementary base pairing. In certain approaches, mismatches or imperfect hybridization may also take place. “Mismatch” typically refers to pairing of noncomplementary nucleotide bases between two different nucleic acid strands (e.g., probe and target) during hybridization. Complementary pairing is commonly accepted to be A-T, A-U, and C-G. Conditions of the local environment, such as ionic strength, temperature, and pH can effect the extent to which mismatches between bases may occur, which may also be termed the “specificity” or the “stringency” of the hybridization. Other factors, such as the length of a nucleotide sequence and type of probe, can also affect the specificity of hybridization. For example, longer nucleic acid probes have a higher tolerance for mismatches than shorter nucleic acid probes. In general, protein nucleic acid probes provide higher specificity than corresponding DNA or RNA probes.

As illustrated in the figures, the presence or absence of target marker 112 in the sample is determined through electrocatalytic techniques. These electrocatalytic techniques allow for the detection of extremely low levels of nucleic acid molecules, such as a target RNA molecule obtained from a biological host. Applications of electrocatalytic techniques are described in further detail in U.S. Pat. Nos. 7,361,470 and 7,741,033, and PCT Application No. PCT/US12/024015, which are hereby incorporated by reference herein in their entireties. A brief description of these techniques, as applied to the current system, is provided below, it being understood that the electrocatalytic techniques are illustrative and non-limiting and that other techniques can be envisaged for use with the other systems, devices and methods of the current system (e.g. FIGS. 4-18).

In the electrocatalytic application of FIGS. 1-3C, the sample is applied to the electrode 102 in a solution. In practice, a redox pair having a first transition metal complex 108 and a second transition metal complex 110 is added to the sample solution. A signal generator or potentiostat is used to apply an electrical potential (voltage) to the electrode 102, causing the first transition metal complex 108 to change oxidative states, due to its close association with the electrode 102 and the probe 106. Electrons can then be transferred to the second transition metal complex 110, creating a current through the electrode 102, through the sample, and back to the signal generator. The current signal is amplified by the presence of the first transition metal complex 108 and the second transition metal complex 110, as will be described below.

The first transition metal complex 108 and the second transition metal complex 110 together form an electrocatalytic reporter system which amplifies the signal. A transition metal complex is a structure composed of a central transition metal atom or ion, generally a cation, surrounded by a number of negatively charged or neutral ligands possessing lone pairs of electrons that can be transferred to the central transition metal. A transition metal complex (e.g., complexes 108 and 110) includes a transition metal element found between the Group IIA elements and the Group IIB elements in the periodic table. In certain approaches, the transition metal is an element from the fourth, fifth, or sixth periods between the Group IIA elements and the Group IIB elements of the periodic table of elements. In certain embodiments, the first transition metal complex 108 and second transition metal complex 110 include a transition metal selected from the group comprising cobalt, iron, molybdenum, osmium, ruthenium and rhenium. In certain embodiments, the ligands of the first transition metal complex 108 and second transition metal complex 110 is selected from the group comprising pyridine-based ligands, phenathroline-based ligands, heterocyclic ligands, aquo ligands, aromatic ligands, chloride (Cl⁻), ammonia (NH₃⁺), or cyanide (CN⁺). In certain approaches, the first transition metal complex 108 is a transition metal ammonium complex. For example, as shown in FIG. 1, the first transition metal complex 108 is Ru(NH₃)₆³⁺. In certain approaches, the second transition metal complex 110 is a transition metal cyanate complex. For example, as shown in FIG. 1, the second transition metal complex is Fe(CN)₆³⁻. In certain approaches, the second transition metal complex 110 is an iridium chloride complex such as IrCl₆²⁻ or IrCl₆³⁻.

In certain applications, if the target molecule 112 is present in the sample solution, the target molecule 112 will hybridize with the probe 106, as shown on the right side of FIG. 1. The first transition metal complex 108 (e.g., Ru(NH₃)₆³⁺) is cationic and accumulates, due to electrostatic attraction forces as the nucleic acid target molecule 112 hybridizes at the probe 106. The second transition metal complex 110 (e.g., Fe(CN)₆³⁻) is anionic and is repelled from the hybridized target molecule 112 and probe 106. A signal generator, such as a potentiostat, is used to apply a voltage signal to the electrode. As the signal is applied, the first transition metal complex 108 is reduced (e.g., from Ru(NH₃)₆³⁺ to Ru(NH₃)₆²⁺). The reduction of the second metal complex 110 (e.g., Fe(CN)₆³⁻) is more thermodynamically favorable, and accordingly, electrons (e⁻) are shuttled from the reduced form of the first transition metal complex 108 to the second transition metal complex 110 to reduce the second transition metal complex (e.g., Fe(CN)₆³⁻ to Fe(CN)₆⁴⁻) and regenerate the original first transition metal complex 108 (e.g., Ru(NH₃)₆³⁺). This catalytic shuttling process allows increased electron flow through the electrode 102 when the potential is applied, and amplifies the measured signal (e.g., a current), when the target molecule 112 is present. When the target molecule 112 is absent from the sample, the measured signal is significantly reduced.

Chart 200 of FIG. 2 depicts representative electrocatalytic detection signals. A signal generator, such as a potentiostat, is used to apply a voltage signal at an electrode, such as electrode 102 of FIG. 1. Electrochemical techniques including, but not limited to cyclic voltammetry, amperometry, chronoamperometry, differential pulse voltammetry, calorimetry, and potentiometry may be used for detecting a target marker. In certain approaches, an applied potential or voltage is altered over time. For example, the potential may be cycled or ramped between two voltage points, such from 0 mV to −300 mV and back to 0 mV, while measuring the resultant current. Accordingly, chart 200 depicts the current along the vertical axis at corresponding potentials between 0 mV and −300 mV, along the horizontal axis. Data graph 202 represent a signal measured at an electrode, such as electrode 102 of FIG. 1, in the absence of a target marker. Data graph 204 represents a signal measured at an electrode, such as electrode 102 of FIG. 1, in the presence of a target marker. As can be seen on data graph 204, the signal recorded in the presence of the target molecule provides a higher amplitude current signal, particularly when comparing peak 208 with peak 206 located at approximately −100 mV. Accordingly, the presence and absence of the marker can be differentiated.

In certain applications, a single electrode or sensor is configured with two or more probes, arranged next to each other, or on top of or in close proximity within the chamber so as to provide target and control marker detection in an even smaller point-of-care size configuration. For example, a single electrode sensor may be coupled to two types of probes, which are configured to hybridize with two different markers. In certain approaches, a single probe is configured to hybridize and detect two markers. In certain approaches, two types of probes may be coupled to an electrode in different ratios. For example, a first probe may be present on the electrode sensor at a ratio of 2:1 to the second probe. Accordingly, the sensor is capable of providing discrete detection of multiple analytes. For example, if the first marker is present, a first discrete signal (e.g., current) magnitude would be generated, if the second marker is present, a second discrete signal magnitude would be generated, if both the first and second marker are present, a third discrete signal magnitude would be generated, and if neither marker is present, a fourth discrete signal magnitude would be generated. Similarly, additional probes could also be implemented for increased numbers of multi-target detection.

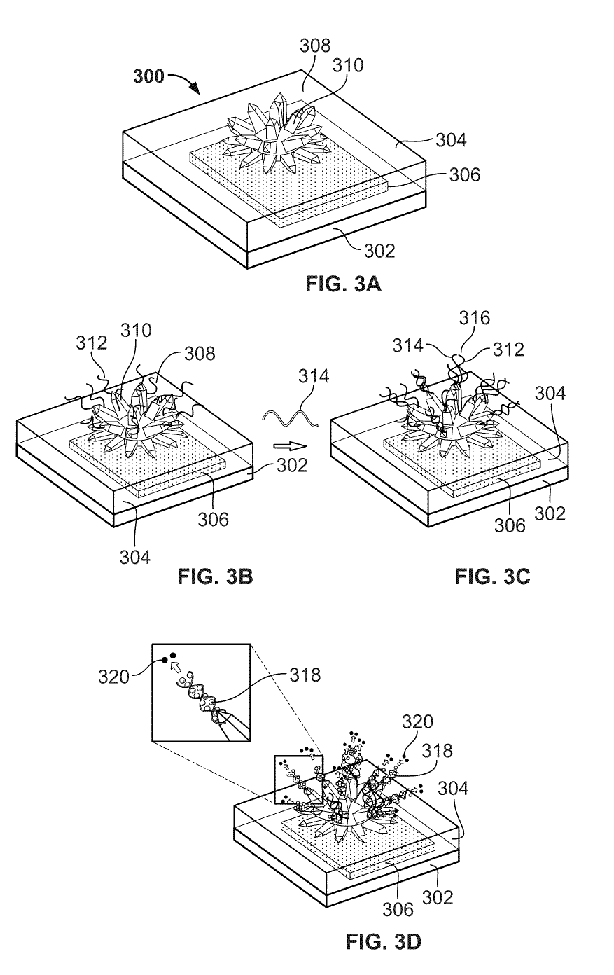

In certain approaches, the systems, devices, and methods described herein include one or more nanostructured microelectrodes (NMEs) that are configured to apply a potential for electrocatalytic detection, as discussed above. FIGS. 3A-3D depict a nanostructured microelectrode system 300 for electrocatalytic detection of a nucleotide strand. Nanostructured microelectrode systems are described in further detail in U.S. application Ser. No. 13/061,465, U.S. Pat. Nos. 7,361,470 and 7,741,033, and PCT Application No. PCT/US12/024015, which are hereby incorporated by reference herein in their entireties. A brief description of nanostructured microelectrodes, as applied to the current system, is provided below.

Nanostructured microelectrodes are microscale electrodes with nanoscale features. In certain approaches, nanostructured microelectrodes have a height in the range of about 0.5 microns (μm) to about 100 μm and a diameter in the range of about 1 μm to about 50 μm. Nanostructured microelectrodes may include additional features, such as spikes, nanowires, and bumps with a morphology in the nanoscale range, for example, between about 1 nanometer (nm) and about 300 nm. In certain approaches, these morphological features are in the range of about 10 nm to about 20 nm. These features may be hemispherical, irregular, cylindrical, or fractal. For example, FIG. 3A depicts a sensor 300 with nanostructured microelectrode 308 having nanowire extensions 310 in a fractal configuration. The nanowire extensions 310 range from about 10 nm to about 80 nm in diameter, and the nanowire extensions 310 have a density range from about 1×10⁸ to about 1×10⁹ nanowire extensions per square centimeter.

In certain approaches, nanostructured microelectrodes are comprised of a noble metal (e.g., gold, platinum, palladium, copper, silver, osmium, indium, rhodium, ruthenium), alloys of noble metals (e.g., gold-palladium, silver-platinum, etc.), conducting polymers (e.g., polypyrole (PPY)), non-noble metals (e.g., nickel, aluminum, tin, titanium, tungsten), metal oxides (e.g., zinc oxide, tin oxide, nickel oxide, indium tin oxide, titanium oxide, nitrogen-doped titanium oxide (TiOxNy)), metal silicides (nickel silicide, platinum silicide), metal nitrides (titanium nitride (TiN), tungsten nitride (WN), or tantalum nitride (TaN)), carbon (nanotubes, fibers, graphene, amorphous carbon), or combinations of any of the above.

Nanostructured microelectrodes are formed on a solid substrate 302. Solid substrate 302 may comprise a semiconductor material, such as silicon, silica, quartz, germanium, gallium arsenide, silicon carbide and indium compounds (e.g., indium arsenide, indium, antimonide, indium phosphide), selenium sulfide, ceramic, glass, plastic, polycarbonate or other polymer or combinations of any of the above. System 300 may be provided in the form of a chip, such as an integrated circuit (IC) chip. In certain approaches, system 300 includes an insulation or dielectric layer 304, which is comprised of a material having high electrical resistance. Examples of appropriate materials include, but are not limited to, silicon dioxide, silicon nitride, nitrogen doped silicon oxide (SiOxNy) or parylene. The nanostructured microelectrode 308 is generally formed by providing a cylindrical pore in insulation layer 304 to a conductive lead 306. Conductive lead 306 may be comprised of gold, silver, tungsten, titanium nitride, polysilicon or other conductive or semiconductive materials. The nanostructured microelectrode 308 is formed by placing the sensor 300 in a solution containing an ionic form of the electrode metal and applying an electrical signal to electrically deposit or grow electrode 308 and the resultant nanostructures, such as nanowire extensions 310.

As shown in FIG. 3B, nanostructured microelectrode 308 can be functionalized with a probe 312, which is similar to probe 106, and may be, for example, a peptide nucleic acid probe. In certain approaches, probe 312 is attached to nanowire extensions 310 with a linker molecule, as described above in relation to linker 104. The surface of nanostructured microelectrode 308 may be further coated with a material which maintains the electrode's high conductivity, but facilitates binding with probe 312. For example, nitrogen containing nanostructured microelectrodes (e.g., TiN, WN, or TaN) can bind with an amine functional group of probe 312.

FIGS. 3C and 3D depict the use of nanostructured microelectrode 308 for electrocatalytic detection of a target marker 314, which is similar to target marker 112. This electrocatalytic detection process is similar to the electrocatalytic detection process of FIG. 1. When present, target marker 314 hybridizes with probe 312 to form a hybridized complex 316. An electrical signal (e.g., a voltage) is applied to electrode 308. A first transition metal complex 318 receives and shuttles electrons to a second transition metal complex 320 to provide an amplified signal when the target molecule 314 is present, as described in relation to FIG. 1 and FIG. 2.

In certain aspects, the sensors and electrodes described herein are integrated into a sensing or analysis chamber, for example in a point-of-care device, to analyze a sample from a biological host. FIG. 4 depicts an analysis chamber 400 with a pathogen sensor 406 and a host sensor 410. The chamber 400 includes walls 402 and 404 that form a space with which a sample is retained and analyzed at sensors 406 and 410. Pathogen sensor 406 includes a conductive trace 408 to connect the sensor 406 to controlling instrumentation such as a potentiostat. Host sensor 410 is also connected to external or controlling instrumentation with a conductive trace 412. Pathogen sensor 406 and host sensor 410 are separated by a distance X₁.

The pathogen sensor 406 and the host sensor 410 may be similar to previously described electrodes and sensors such as electrode 102 and nanostructured microelectrode 308. The pathogen sensor 406 is used to determine whether or not the marker is present in the sample. Although not depicted in FIG. 4, pathogen sensor 406 includes a probe, such as probe 106, configured to couple to a target marker from a pathogen. In certain approaches, the probe is a peptide nucleic acid probe. For example the probe coupled to the pathogen sensor 406 may include a nucleotide sequence that is complementary to a nucleotide sequence from a pathogen which is unique to that pathogen. In certain approaches, the probe is configured to couple to an RNA molecule from Chlamydia trachomatis. Example probes for identifying the presence of a target marker for Chlamydia trachomatis include, but are not limited to, probes for 16s rRNA with sequences of CGTTACTCGGATGCCCAAAT or ATCTTTGACAACTAACTTAC, probes for 23s rRNA with sequences of CTTGACCCTTACGGGCCATT or TTCTCATCGCTCTACGGACT, probes for CTLon_{—}0332 with sequences of ATATACACCCAGGCTCCC or GCCTAACCGCTCAGTGATAA, and probes for omcA with sequences of TACGACAACAACTACTTAAA, AGCATCTTGGTGCGTATCCC, or TGCATTTGCCGTCAACTGGA.

The host sensor 410 includes a probe configured to couple to a host marker. The host marker is an endogenous element from a biological host, such as a DNA sequence, RNA sequence, or peptide. For example, the probe coupled to host sensor 410 may be configured with a nucleotide sequence that hybridizes with a nucleotide sequence unique to the human genome. In certain approaches, the probe for the host marker is a peptide nucleic acid probe. Preferably, the host marker is present in every biological sample taken from the human patient, and therefore can serve as a positive, internal control for the analysis process. Accordingly, detection of the host marker at host sensor 410 serves as a control for the assay. Specifically, detection of the host marker confirms that the sample was taken correctly from the host (e.g., a patient), that the sample was processed correctly, and that hybridization of the probe and marker in the analysis chamber has taken place successfully. If any part of the assay fails, and the host marker is not detected at host sensor 410, the assay is considered indeterminate.

The pathogen sensor 406 and host sensor 410 operate using the electrocatalytic methods described previously in relation to FIGS. 1-3 (although such sensors and the internal control techniques discussed herein could also be applied in other diagnostic methods). FIG. 4 depicts only two sensors, but any number of sensors may be used. For example, chamber 400 may include a plurality of pathogen sensors 406 and a plurality of host sensors 410. When a plurality of sensors is used, each sensor may optionally be configured to sense a different target marker in order to detect the presence or absence of different pathogens, different hosts, or different parts of the same pathogen or the same host. In alternative approaches, a plurality of pathogen sensors 406 is used, but each pathogen sensor is configured to sense the same target marker in order to provide additional verification of the presence or absence of that target marker. Similarly, a plurality of host sensors 410 may also be used with each sensor being configured to detect the presence or absence of the same host target marker to provide additional verification of the measurement.

FIG. 5 depicts an additional embodiment of an analysis chamber. Chamber 500 is similar to chamber 400 in that it includes walls 402 and 404, pathogen sensor 406 and host sensor 410. Chamber 500 additionally includes a non-sense sensor 414. Similar to pathogen sensor 406 and host sensor 410, non-sense sensor 414 is electrically coupled to controlling instrumentation, such as a potentiostat, with a conductive trace 416. The non-sense sensor 414 may also include an electrode, such as a nanostructured microelectrode. Non-sense sensor 414 includes a probe, such as probe 106. In certain approaches, the non-sense probe is a peptide nucleic acid probe. The non-sense probe, however, is not configured to mate with a marker from the pathogen or the biological host. Instead, the probe coupled to non-sense sensor 414 has a structure, such as a nucleotide sequence, which is not found in either the pathogen or the biological host. The non-sense sensor serves as an additional control to verify that the conditions within analysis chamber 500 can provide accurate sensing results. Non-sense sensor 414 tests for nonspecific binding. Nonspecific binding of a nucleotide sequence may occur under inappropriate hybridization conditions in chamber 500. For example, nonspecific binding may occur when the pH, ionic strength, or temperature are not appropriate for accurate testing. If binding occurs at non-sense sensor 414, then other nonspecific binding may take place at pathogen sensor 406 and the host sensor 410, and therefore the assay would be inaccurate. The non-sense sensor 414 is thereby able to act as an additional control for testing conditions. The non-sense sensor 414 may also function using electrocatalytic techniques as previously described. Although FIG. 5 depicts three sensors, any number of sensors could be used. Sensors 406, 410, and 414 are arranged in chamber 500 in a linear arrangement. However, sensors 406, 410, and 414 may also be arranged in other patterns.

FIG. 6 depicts an additional embodiment of an analysis chamber 600 which is similar to chambers 400 and 500 previously described. FIG. 6 also depicts a reference electrode 418 and a counter electrode 422. The reference electrode 418 and counter electrode 422 are connected to the controlling instrumentation (e.g., a potentiostat) by conductive traces 420 and 424, respectively. The reference electrode 418 and counter electrode 422 are used in the electrocatalytic measurements. The reference electrode 418 serves as a reference for applying a voltage at any of the sensors 406, 410, and 414. When a voltage is applied at a sensor (e.g., sensors 406, 410, and 414), the current generated flows through sensor (e.g., sensors 406, 410, and 414), through the hybridized complex of the probe and target, through the sample, and through the counter electrode 422.

FIG. 7 depicts a system for analyzing a biological sample that is configured to be applied as a biosensor system similar to those shown in FIGS. 4-6. System 700 includes an inlet channel 702 coupled to an analysis chamber 704 which is coupled to an outlet channel 706. Analysis chamber 704 is similar to previously discussed analysis chambers 400, 500, and 600. Analysis chamber 704 includes two pathogen sensors 406, two host sensors 410, and two non-sense sensors 414. Although two sensors of each type are depicted, any number of sensors may be used in the chamber 704. Analysis chamber 704 additionally includes reference electrode 418 and counter electrode 422 that function as described in relation to FIG. 6.

System 700 is configured to allow flow of a sample solution into contact with one or more biosensors. Inlet channel 702 has a diameter d₁. Analysis chamber 704 has a diameter d₂. Outlet chamber 706 has a diameter d₃. In certain approaches, the diameters d₁, d₂, and d₃ are substantially similar. In certain embodiments, the diameters d₁, d₂, and d₃ are between approximately 25 mm and 3 mm. When the sample flows in system 700 through inlet channel 702, chamber 704, and outlet channel 706, the flow can be maintained at a constant rate. A constant, even flow, which can be laminar in certain cases, may be particularly desirable to ensure that the sample is exposed to each electrode or sensor for approximately the same amount of time. In certain approaches, the diameters d₁, d₂, and d₃ are substantially different. Different diameters may be used in the ends and mid-sections of the channel 702 to adjust flow rates. For example, a fast flow rate may be desirable through the inlet channel to move the sample to the chamber, but a slower flow rate may be desirable when the sample is within the chamber 704 so as to provide longer exposure time. For example, the diameter d₁ could be smaller than the mid-section or diameter d₂ of the chamber.

Different diameters d₁, d₂, and d₃ may also be useful to adjust for differences in volume of the sample. For example, in certain approaches, a reagent may be added to the sample as it travels through a part of system 700. A wider diameter may be used in that portion to compensate for the increased volume, and in certain approaches, to maintain a particular flow rate of the sample as the reagent is added to the sample.

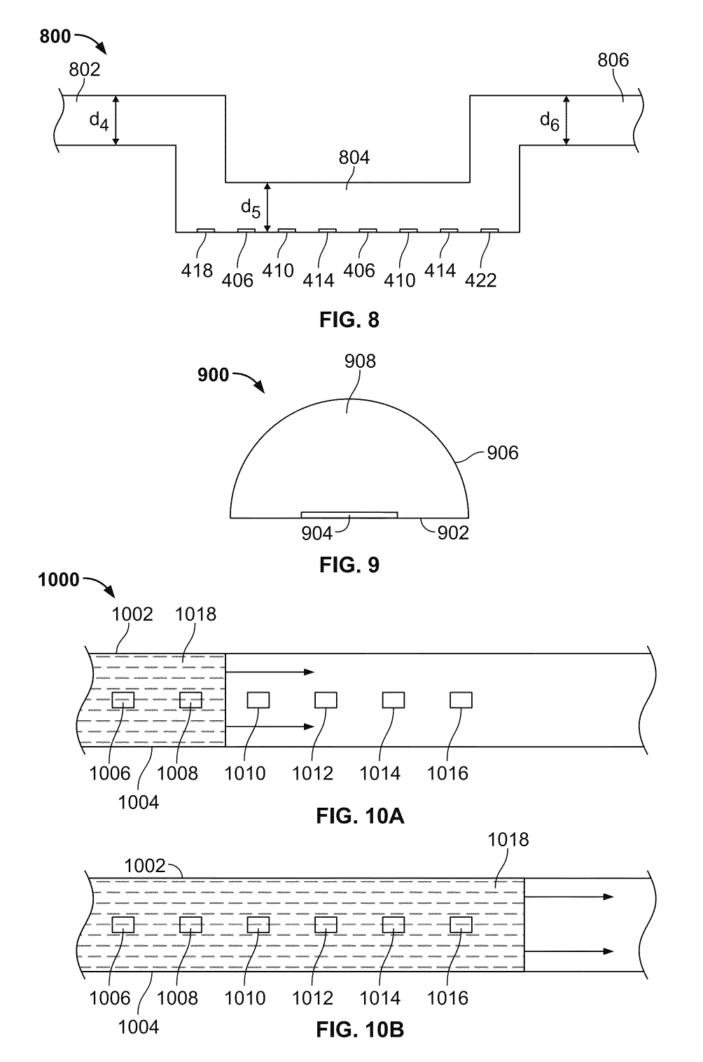

As shown, the inlet channel 702, analysis chamber 704, and outlet channel 706 are arranged in a linear manner, but the inlet channel 702, analysis chamber 704, and outlet channel 706 may include curves, turns, or may be arranged at different heights or depths. For example, FIG. 8 depicts a system 800 with an inlet channel 802, analysis chamber 804 and outlet channel 806 positioned at different levels. The inlet channel 802 and outlet chamber 806 are higher than the analysis chamber 804, which may allow portions of the sample solution for analysis within chamber 802 to be separated from other portions, such as waste. The diameters d₄, d₅, and d₆, respectively, of the inlet channel 802, analysis chamber 804, and outlet channel 806 may also be substantially similar or substantially different as described in relation to system 700 of FIG. 7. Analysis chamber 804 may be positioned at a different level than inlet channel 802 or outlet channel 806 for convenience in manufacturing. For example, the sensors may be manufactured separately from the inlet channel 802 and outlet channel 806 and later positioned within the chamber 804. Inlet channel 802 and outlet channel 806 may be manufactured from a mold, while the sensors 406, 410, 414 and electrodes 418 and 422 may be manufactured on a silicon support or printed circuit board and attached to the inlet channel 802 and outlet channel 806.

FIG. 9 depicts a cross-sectional view of an embodiment of an analysis chamber. The chamber 900 includes a base portion 902, one or more sensors 904, and a wall 906 that extends above the base in an arc. The wall 906 is coupled to the base 902 to form a retaining space 908 within which the sample is retained and flows. As shown, the space 908 is shaped like a half-pipe with a substantially flat base and arced roof. In certain approaches, the wall 906 is manufactured separate from the base 902 and electrode 904. For example the wall 906 may be manufactured through a molding technique, and the base 902, and sensor 904 may be manufactured with integrated circuit technology. The wall 906 may then be coupled to the base 902 to form the chamber 900.

FIGS. 10A-10B depict flow of a sample through an analysis chamber. The chamber 1000 includes at least one sensor configured for detecting a target component and a control maker in a sample. The at least one sensor includes probes that are specific to the target and control marker, respectively. Chamber 1000 is similar to previously depicted analysis chambers. Chamber 1000 includes walls 1002 and 1004 and a plurality of sensors 1006, 1008, 1010, 1012, 1014, 1016 similar to sensors 406, 410, 414 of FIGS. 4-8. In certain approaches, the one or more sensors include one or more electrodes with probes, such as one or more nanostructured microelectrodes, as previously described, configured with nucleotide sequence probes (e.g., PNAs) that can bind to target and control components in the sample. The sensors can be configured as pathogen sensors for detecting a target marker (e.g., indicative of Chlamydia trachomatis), host sensors for detecting a control marker (e.g., indicative of human tissue), non-sense sensors with a non-sense probe, or any combination thereof. In certain approaches, at least one sensor is configured as a pathogen sensor and at least one sensor is configured as a host sensor. At least one sensor may also be configured as a non-sense sensor. Chamber 1000 may include a plurality of any or each sensor. For example, sensor 1006 and 1012 may be configured as pathogen sensors, sensors 1008 and 1014 may be configured as host sensors, and sensors 1010 and 1016 may be configured as non-sense sensors. Chamber 1000 may also include reference electrodes and counter electrodes, such as reference electrode 418 and counter electrode 422.

Sample 1018 flows through the chamber 1000, analytes, and in particular, target markers and control markers, can hybridize with probes on the sensors. In certain approaches, sample 1018 flows at a constant flow rate through chamber 1000. In certain approaches, each sensor is exposed to the sample for approximately the same amount of time. In certain approaches, the sample 1018 is agitated in chamber 1000 to improve or accelerate hybridization.

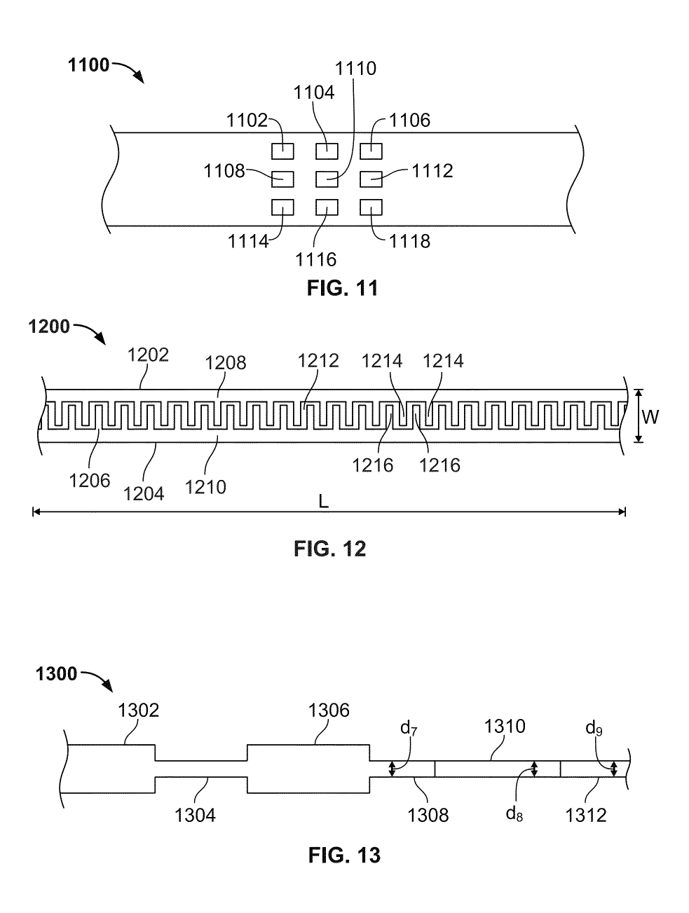

Sensors 1006, 1008, 1010, 1012, 1014, and 1016 are depicted in a linear arrangement in FIG. 10. In certain approaches, other arrangements may be used. For example, FIG. 11 depicts a chamber 1100 with sensors 1102, 1104, 1106, 1108, 1110, 1112, 1114, 1116, and 1118 positioned in an array or grid configuration. A grid arrangement may be helpful to provide increased numbers of sensors or to shorten the chamber. In certain approaches, each sensor type (e.g., pathogen sensor, host sensor, and non-sense sensor) occupies a row of a grid configuration to ensure similar exposure to the sample for each sensor type. In certain approaches, each sensor type (e.g., pathogen sensor, host sensor, and non-sense sensor) occupies a column of a grid configuration. In certain approaches, each sensor type (e.g., pathogen sensor, host sensor, and non-sense sensor) alternates along each row and each column of a grid configuration.

A biological sample may be processed to release, or otherwise make accessible, the target molecules or analytes of interest, such as the target marker and control marker. For example, analytes, such as nucleic acids, may normally be sequestered inside of cells, bacteria, or viruses from which they need to be released prior to characterization. For example, mechanical approaches including, but not limited to, sonication, centrifugation, shear forces, heat, and agitation may be used to process a biological sample. Additionally or alternatively, chemical methods including, but not limited to, surfactants, chaotropes, enzymes, or heat may be applied to produce a chemical effect.

In certain approaches, lysis techniques are applied to a biological sample to release target markers from cells within the sample. Lysis techniques disrupt the integrity of a biological compartment such as a cell such that internal components, such as RNA, are exposed to and may enter the external environment. Lysis procedures may cause the formation of permanent or temporary openings in a cell membrane or complete disruption of the cell membrane, to release cell contents into the surrounding solution. For example, a modulated electrical potential can be applied to a sample to release nucleic acids, and in particular, RNA, into the sample solution. Electrical lysis techniques are described in further detail in PCT Application No. PCT/US12/28721, which is hereby incorporated by reference herein in its entirety. A brief description of these techniques, as applied to the current system, is provided below.

FIG. 12 depicts an electrical lysis chamber. Chamber 1200 includes a first wall 1202 and a second wall 1204 defining a space 1206 in which a sample is retained. For example, a sample may flow through the space 1206 of the lysis chamber 1200. Chamber 1200 also includes a first electrode 1208 and second electrode 1210. First electrode 1208 and second electrode 1210 are electrically independent and separated by a spacing 1212.

First electrode 1208 and second electrode 1210 are composed of a conductive material. For example, first electrode 1208 and second electrode 1210 may comprise carbon or metals including, but not limited to, gold, silver, platinum, palladium, copper, nickel, aluminum, ruthenium, and alloys thereof. First electrode 1208 and second electrode 1210 may comprise conductive polymers, including, but not limited to polypyrole, iodine-doped trans-polyacetylene, poly(dioctyl-bithiophene), polyaniline, metal impregnated polymers and fluoropolymers, carbon impregnated polymers and fluoropolymers, and admixtures thereof. In certain embodiments, first electrode 1208 and second electrode 1210 comprise a combination of these materials.

In certain embodiments, the spacing 1212 separates the first electrode 1208 and the second electrode 1210 by a range of approximately 1 nm to approximately 2 mm. In certain embodiments, the first electrode 1208 and the second electrode 1210 are interdigitated electrodes. For example, the first electrode 1208 may have digits 1214 spaced between digits 1216 of the second electrode 1210. The spacing 1212 can be composed of an insulating material to further localize the applied potential difference to the electrodes. For example, spacing 1212 may comprise silicon dioxide, silicon nitride, nitrogen doped silicon oxide (SiOxNy), parylene, or other insulating or dielectric materials.

As shown, first electrode 1208 and second electrode 1210 are planar electrodes, over which the sample flows. For example, first electrode 1208, second electrode 1210, and spacing 1212 are coplanar to form a base within space 1206 of the chamber 1200. First electrode 1208 and second electrode 1210 may also comprise other configurations, including, but not limited to, arrays, ridges, tubes, and rails. First electrode 1208 and second electrode 1210 may be positioned on any portion of chamber 1200, including, but not limited to sides, bottom surfaces, upper surfaces, and ends. The lysis chamber 1200, first electrode 1208, second electrode 1210, and spacing 1212 may have any appropriate length L. Although depicted as having the same length L in FIG. 12, each component of the chamber 1200 may have a different length. In certain approaches, the length L of the chamber 1200 is between approximately 0.1 mm and 100 mm. For example, the chamber 1200 may have a length L of approximately 50 mm. Similarly, the lysis chamber 1200, first electrode 1208, second electrode 1210, and spacing 1212 may have any appropriate width W. Each component of the chamber may have a different width. In certain approaches, the width W of the chamber 1200 is between approximately 0.1 mm and 10 mm. For example, the chamber 1200 may have a width W of 2 mm. The chamber 1200 is depicted as linear or straight, however, in certain approaches, the chamber 1200 includes turns, bends, and other nonlinear structures. FIG. 12 depicts two electrodes 1208 and 1210, but any number of electrodes may be used. In embodiments comprising more than two electrodes, the electrodes may be electrically independent to enable applying different potentials between different electrodes. A plurality of electrodes may be arranged in other configurations, such as arrays.

A modulated electrical potential may be applied to first electrode 1208 and second electrode 1210 when in contact with a biological sample to release and controllably fragment nucleic acids from biological compartments (e.g., cells, bacteria, etc.) within the sample into the sample solution. The applied potential may be modulated in a variety of ways in order to induce lysis of biological compartments within a sample. In certain embodiments, a voltage ranging from about 0.5V to about 3,000V is applied between first electrode 1208 and second electrode 1210. In a certain embodiments, the voltage applied between first electrode 1208 and second electrode 1210 is about 40V. This voltage may be constant or may be applied in pulses. In certain approaches, the duration of such voltage pulses is up to about 60 seconds. In certain approaches, the duration of a voltage pulse or pulse width is about 30 milliseconds. The interpulse interval or time between voltage pulses is between about 0.1 seconds and 360 seconds. In certain embodiments, the interpulse interval is about 1 second. A voltage pulse may be applied to the first electrode 1208 and second electrode 1210 as a repeating waveform. Voltage waveforms include, but are not limited to, triangle waves, square waves, sine waves, exponential decaying waves, forward saw tooth waveforms, and reverse saw tooth waveforms. For example, the voltage pulse may be a square wave. The voltage pulse may have any appropriate frequency. In certain embodiments, the pulse has a frequency between about 0.1 Hz and 1 kHz. For example, the voltage pulse may have a frequency of 1 Hz. In certain approaches, the lysis pulses are applied as the sample continuously flows through chamber 1200. Lysis pulses may also be applied while the sample is immobile in the chamber, or during agitation of the sample. The total application time of the pulses is between about 1 second and 1000 seconds. In certain approaches, the pulses are applied for 2 minutes. In certain approaches, the pulses are applied for 20 seconds.

In certain embodiments, the electrically-based lysis procedure controllably fragments analyte molecules, such as DNA and RNA. Fragmentation can advantageously reduce the time required to detect or otherwise characterize the released analyte. For example, fragmentation of an analyte molecule may reduce molecular weight and increase speed of diffusion, thereby enhancing molecular collision and reaction rates. In another example, fragmenting a nucleic acid may reduce the degree of secondary structure, thereby enhancing the rate of hybridization to a complementary probe molecule. For example, RNA from a cell lysed by the application of a modulated potential to first electrode 1208 and second electrode 1210 may have an average length of over 2,000 bases immediately upon lysis, but are rapidly cleaved into fragments of reduced length under continued lysing conditions. The average size of such fragments may be up to about between about 20% and about 75% of the size or length of the unfragmented analyte. In certain approaches, the analyte is RNA. For example, fragmented RNA may have a significant portion of molecules with lengths between approximately 20 and approximately 500 bases. In certain approaches, pulses are modulated to simultaneously lyse and fragment the sample and analytes. Additionally or alternatively, a second set of electrical pulses may be applied and configured to provide specific, controlled fragmentation. For example, a first set of pulses may be applied to provide lysis, and a second set of pulses may be applied to provide fragmentation. In certain approaches, the first pulse set for lysis and second pulse set for fragmentation are alternated.

Fragmentation is controllably adjusted by changing the pulse parameters (magnitude, frequency, interpulse interval, pulse width, application time, etc.) as described above. Accordingly, subsequent hybridization times of a target marker at a probe can be controlled and reduced compared to hybridization times for unfragmented nucleic acids under otherwise similar conditions. For example, hybridization times may be reduced by between about 25% and about 80%. Accordingly, the time required for electrochemical analysis, for example at sensors 102, 308, 406, and 410 may also be reduced.

FIG. 13 depicts a system for preparing and analyzing a biological sample. System 1300 may include a receiving chamber 1302, a first channel, 1304, a lysis chamber 1306, a second channel 1308, an analysis chamber 1310, and a third channel 1312. Other processing chambers and channels may also be included. In practice, a user obtains a sample from a biological host and places the sample in receiving chamber 1302. While in receiving chamber 1302, the sample may undergo processing, such as filtering to remove undesirable matter, addition of reagents, and removal of gases. The sample is then moved from receiving chamber 1302 through channel 1304 and into lysis chamber 1306. The sample may be moved by applying external pressure with fluids or gases, for example, with a pump or pressurized gas. In certain approaches, lysis chamber 1306 is similar to lysis chamber 1200 of FIG. 12. The sample undergoes a lysis procedure, such as an electrical lysis procedure, as described previously. The lysis procedure may also cause fragmentation of the analytes, such as RNA, which serve as target markers and control markers. The sample is then moved through channel 1308 into analysis chamber 1310. Analysis chamber 1310 may be similar to previously described analysis chambers 400, 500, 600, 700, 800, 900, 1000, and 1100. Analysis chamber 1310 includes one or more sensors, such as pathogen sensors, host sensors, and non-sense sensors. The target markers and control markers can hybridize with probes on their respective sensors. The presence of the target markers and control markers are analyzed at the sensors, for example, with electrocatalytic techniques, as described previously in relation to FIGS. 1-3. In certain approaches, the sample is then pumped through channel 1312 to additional processing, storage, or waste areas.

The dimensions, such as lengths, widths, and diameters of the sections of system 1300 can be configured to adjust for different volumes, flow rates, or other parameters. FIG. 13 depicts channel 1308 with diameter d₇, analysis chamber 1310 with diameter d₈, and channel 1312 with diameter d₉. In certain approaches, diameters d₇, d₈, and d₉ are each approximately the same to provide an even flow into and through analysis chamber 1310. In certain approaches, diameters d₇, d₈, and d₉ have different sizes to accommodate for different flow rates, the addition of reagents, or removal of portions of the sample.

In certain approaches, the systems, devices, and methods described herein are used for diagnosing a disease in a human. The systems, devices, and methods may be used to detect bacteria, viruses, fungi, prions, plant matter, animal matter, protein, RNA sequences, DNA sequences, cancer, genetic disorders, and genetic traits. For example, Chlamydia is a bacterial infection caused by the bacteria Chlamydia trachomatis. A caretaker, such as a nurse or physician, may obtain a sample from a patient desiring to receive a diagnosis for this infection. For example, the caretaker may use a medical swab to wipe a surface of the vagina, to thereby obtain a biological sample of vaginal fluid and vaginal epithelial cells. If the patient is carrying the Chlamydia trachomatis bacteria, the bacteria would be present in the sample. Additionally, markers specific to the human genome would also be present. The caretaker or technician may then use the systems, devices, and methods described herein to detect the presence or absence of the bacteria or other pathogen, cell, protein, or gene.

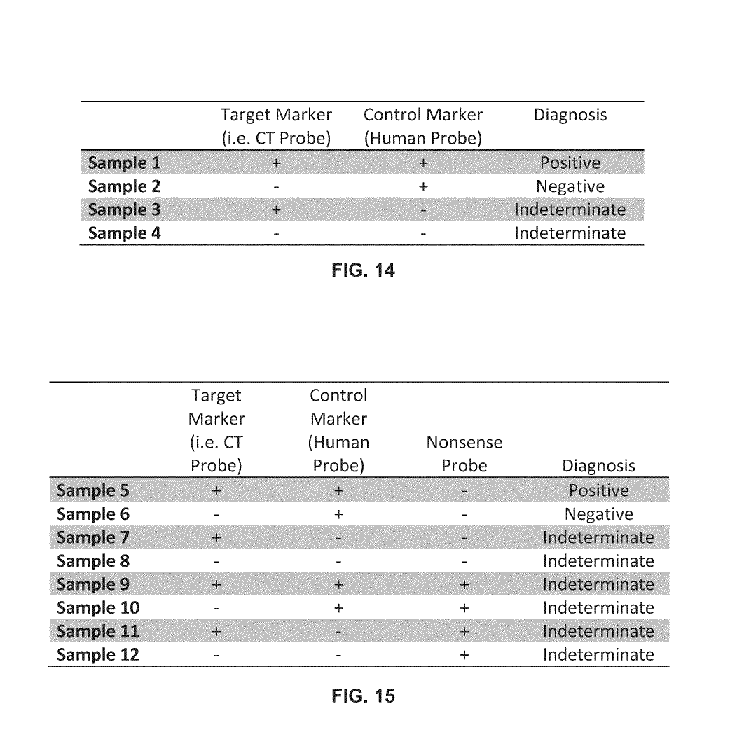

FIG. 14 depicts an interpretation table for application of a two-probe system. A first probe serves as a pathogen sensor and is configured with a probe to detect a target marker, such as a specific RNA sequence for Chlamydia trachomatis (CT). A second probe serves as a host sensor or control, and is configured with a probe to detect a host marker, such as an RNA or DNA sequence from vaginal epithelial cells. The host marker is expected to be present in the biological sample. In fact, the host marker is preferably selected on the basis of being present endogenously in every sample and therefore can serve as a positive control for the analysis process. Accordingly, detection of the host marker serves as a control for three aspects of the assay. Specifically, detection of the host marker confirms that the sample was taken correctly from the patient, that the lysis and fragmentation procedure was performed successfully, and that the hybridization in the analysis chamber has occurred successfully. If any part of the assay fails, and the host marker is not detected at the host sensor, the assay is considered indeterminate.

The two-probe system has outcomes depicted by Sample 1, Sample 2, Sample 3, and Sample 4 in FIG. 14. A positive sign (+) indicates a positive detection result or that the marker was detected in the sample, and a negative sign (−) indicates a negative detection result, no detection of the marker, or absence of the marker in the sample. Sample 1 shows a positive detection result for the target marker (CT), indicating the presence of CT in the sample, and a positive detection result for the control marker, indicating that the sample was appropriately obtained and processed. Sample 1, therefore, would be considered a positive diagnosis of Chlamydia. Sample 2 shows a negative detection result for the target marker (CT), indicating the absence of CT in the sample, and a positive detection result for the control marker, indicating that the sample was appropriately obtained and processed. Sample 2, therefore, would be considered a negative diagnosis of Chlamydia or a detection of the absence of Chlamydia. Sample 3 and Sample 4 both show a negative detection result for the control marker, indicating that the sample was either not obtained correctly or not processed correctly, and the results are therefore indeterminate.

In certain implementations, the sensors of the system of FIG. 14 are configured for use in a biosensor, such as an electrocatalytic sensor with one or more NMEs, as described in FIGS. 1-8. At least one sensor is an NME with a probe having a sequence specific for CT and a second probe specific for a sequence from a vaginal epithelial cell. Multiple NMEs can be used, of course, with a first NME tethered to the CT probe and a second NME tethered to the epithelial cell probe. Measuring current through the CT control NME identifies the presence or absence of CT, and measuring current through the control probe NME identifies the presence or absence of the epithelial or other control component. Other sensor systems can also be used to detect the presence or absence of internal control and target markers from the same sample.

FIG. 15 depicts an interpretation table for application of a three-probe system. Similar to FIG. 14, a first probe serves as a pathogen sensor and is configured with a probe to detect a target marker, such as a specific RNA sequence for Chlamydia trachomatis (CT). A second probe serves as a host sensor or control, and is configured with a probe to detect a host marker, such as an RNA or DNA sequence from vaginal epithelial cells. The third probe is a non-sense sensor with a non-sense probe. The non-sense probe, however, is not configured to mate with a marker from the pathogen or the biological host. Instead, the non-sense probe has a sequence which is not found in either the pathogen or the biological host. The non-sense sensor serves as an additional control to verify that the conditions within the sensing chamber can provide accurate results. A positive result at the non-sense probe may indicate nonspecific hybridization at the non-sense probe, and accordingly other nonspecific hybridization may occur at the other probes. Therefore, a positive result at the non-sense sensor indicates that the results at the other sensors are not reliable, and the overall result is indeterminate.

The three-sensor system has outcomes depicted by Sample 5, Sample 6, Sample 7, Sample 8, Sample 9, Sample 10, Sample 11, and Sample 12 in FIG. 15. Sample 5 shows a positive detection result for the target marker (CT), indicating the presence of CT in the sample, a positive detection result for the control marker, indicating that the sample was appropriately obtained and processed and a negative detection result at the non-sense sensor indicating normal hybridization. Sample 5, therefore, would be considered a positive diagnosis of Chlamydia. Sample 6 shows a negative detection result for the target marker (CT), indicating the absence of CT in the sample, a positive detection result for the control marker, indicating that the sample was appropriately obtained and processed and a negative detection result at the non-sense sensor indicating normal hybridization. Sample 6, therefore, would be considered a negative diagnosis of Chlamydia. Sample 7, Sample 8, and Sample 11 show a negative detection result for the control marker, indicating that the sample was either not obtained correctly or not processed correctly, and the results are therefore indeterminate. Sample 9, Sample 10, Sample 11, and Sample 12 show a positive detection result for the non-sense probe, indicating that nonspecific hybridization has occurred, and the results are therefore indeterminate.

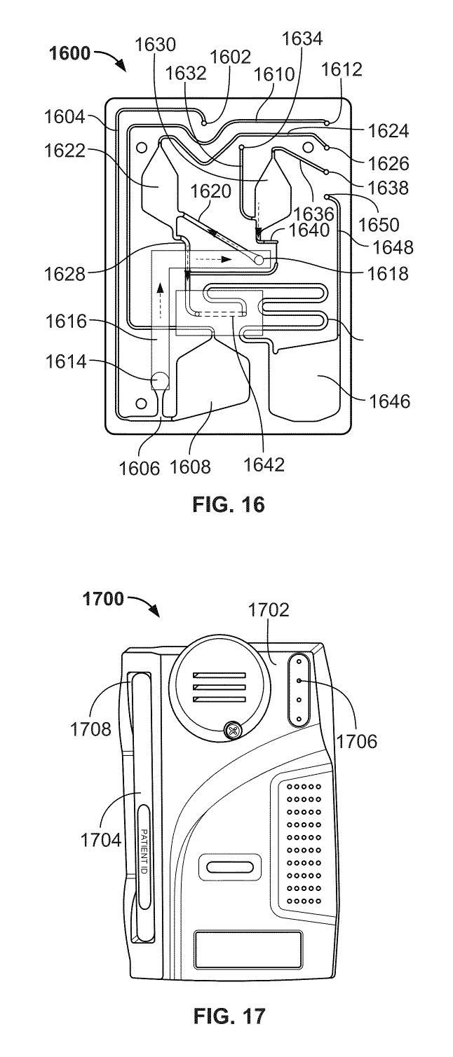

The systems, devices, methods, and electrode and lysis zone embodiments described above may be incorporated into a cartridge to prepare a sample for analysis and perform a detection analysis. FIG. 16 depicts a cartridge system 1600 for receiving, preparing, and analyzing a biological sample. For example, cartridge system 1600 may be configured to remove a portion of a biological sample from a sample collector or swab, transport the sample to a lysis zone where a lysis and fragmentation procedure are performed, and transport the sample to an analysis chamber for determining the presence of various markers and to determine a disease state of a biological host.

The system 1600 includes ports, channels, and chambers. System 1600 may transport a sample through the channels and chambers by applying fluid pressure, for example, with a pump or pressurized gas or liquids. In certain embodiments, ports 1602, 1612, 1626, 1634, 1638, and 1650 may be opened and closed to direct fluid flow. In use, a sample is collected from a patient and applied to the chamber through port 1602. In certain approaches, the sample is collected into a collection chamber or test tube, which connects to port 1602. In practice, the sample is a fluid, or fluid is added to the sample to form a sample solution. In certain approaches, additional reagents are added to the sample. The sample solution is directed through channel 1604, past sample inlet 1606, and into degassing chamber 1608 by applying fluid pressure to the sample through port 1602 while opening port 1612 and closing ports 1626, 1634, 1638, and 1650. The sample solution enters and collects in degassing chamber 1608. Gas or bubbles from the sample solution also collect in the chamber and are expelled through channel 1610 and port 1612. If bubbles are not removed, they may interfere with processing and analyzing the sample, for example, by blocking flow of the sample solution or preventing the solution from reaching parts of the system, such as a lysis electrode or sensor. In certain embodiments, channel 1610 and port 1612 are elevated higher than degassing chamber 1608 so that the gas rises into channel 1610 as chamber 1608 is filled. In certain approaches, a portion of the sample solution is pumped through channel 1610 and port 1612 to ensure that all gas has been removed.

After degassing, the sample solution is directed into lysis chamber 1616 by closing ports 1602, 1634, 1638, and 1650, opening port 1626, and applying fluid pressure through port 1612. The sample solution flows through inlet 1606 and into lysis chamber 1616. In certain approaches, system 1600 includes a filter 1614. Filter 1614 may be a physical filter, such as a membrane, mesh, or other material to remove materials from the sample solution, such as large pieces of tissue, which could clog the flow of the sample solution through system 1600. Lysis chamber 1616 may be similar to lysis chamber 1200 or lysis chamber 1310 described previously. When the sample is in lysis chamber 1616, a lysis procedure, such as an electrical lysis procedure as described above, may be applied to release analytes into the sample solution. For example, the lysis procedure may lyse cells to release nucleic acids, proteins, or other molecules which may be used as markers for a pathogen, disease, or host. In certain approaches, the sample solution flows continuously through lysis chamber 1616. Additionally or alternatively, the sample solution may be agitated while in lysis chamber 1616 before, during, or after the lysis procedure. Additionally or alternatively, the sample solution may rest in lysis chamber 1616 before, during, or after the lysis procedure.

Electrical lysis procedures may produce gases (e.g., oxygen, hydrogen), which form bubbles. Bubbles formed from lysis may interfere with other parts of the system. For example, they may block flow of the sample solution or interfere with hybridization and sensing of the marker at the probe and sensor. Accordingly, the sample solution is directed to a degassing chamber or bubble trap 1622. The sample solution is directed from lysis chamber 1616 through opening 1618, through channel 1620, and into bubble trap 1622 by applying fluid pressure to the sample solution through port 1612, while keeping port 1626 open and ports 1602, 1634, 1638, and 1650 closed. Similar to degassing chamber 1608, the sample solution flows into bubble trap 1622 and the gas or bubbles collect and are expelled through channel 1624 and port 1626. For example, channel 1624 and port 1626 may be higher than bubble trap 1622 so that the gas rises into channel 1624 as bubble trap 1622 is filled. In certain approaches, a portion of the sample solution is pumped through channel 1624 and port 1626 to ensure that all gas has been removed.

After removing the bubbles, the sample solution is pumped through channel 1628 and into analysis chamber 1642 by applying fluid pressure through port 1626 while opening port 1650 and closing ports 1602, 1612, 1634, and 1638. Analysis chamber 1642 is similar to previously described analysis chambers, such as chambers 400, 500, 600, 700, 800, 900, 1000, 1100, and 1306. Analysis chamber 1642 includes sensors, such as a pathogen sensor, host sensor, and non-sense sensor as previously described. In certain approaches, the sample solution flows continuously through analysis chamber 1642. Additionally or alternatively, the sample solution may be agitated while in analysis chamber 1642 to improve hybridization of the markers with the probes on the sensors. In certain approaches, system 1600 includes a fluid delay line 1644, which provides a holding space for portions of the sample during hybridization and agitation. In certain approaches, the sample solution sits idle while in analysis chamber 1642 as a delay to allow hybridization.

System 1600 includes a regent chamber 1630, which holds electrocatalytic reagents, such as transition metal complexes Ru(NH₃)₆³⁺ and Fe(CN)₆³⁻, for electrocatalytic detection of markers in the sample solution. In certain approaches, the electrocatalytic reagents are stored in dry form with a separate rehydration buffer. For example, the rehydration buffer may be stored in a foil pouch above rehydration chamber 1630. The pouch may be broken or otherwise opened to rehydrate the reagents. In certain approaches, a rehydration buffer may be pumped into rehydration chamber 1630. Adding the buffer may introduce bubbles into chamber 1630. Gas or bubbles may be removed from rehydration chamber 1630 by applying fluid pressure through port 1638, while opening port 1634 and closing ports 1602, 1624, 1626, and 1650 so that gas is expelled through channel 1630 and port 1634. Similarly, fluid pressure may be applied through port 1634 while opening port 1638. After the sample solution has had sufficient time to allow the markers to hybridize to sensor probes in the analysis chamber, the hydrated and degassed reagent solution is pumped through channel 1640 and into analysis chamber 1642 by applying fluid pressure through port 1638, while opening port 1650 and closing all other ports. The reagent solution pushes the sample solution out of analysis chamber 1642, through delay line 1644, and into waste chamber 1646 leaving behind only those molecules or markers which have hybridized at the probes of the sensors in analysis chamber 1642. In certain approaches, the sample solution may be removed from the cartridge system 1600 through channel 1648, or otherwise further processed. The reagent solution fills analysis chamber 1642. In certain approaches, the reagent solution is mixed with the sample solution before the sample solution is moved into analysis chamber 1642, or during the flow of the sample solution into analysis chamber 1642. After the reagent solution has been added, an electrocatalytic analysis procedure to detect the presence or absence of markers is performed as previously described.

FIG. 17 depicts an embodiment of a cartridge for an analytical detection system. Cartridge 1700 includes an outer housing 1702, for retaining a processing and analysis system, such as system 1600. Cartridge 1700 allows the internal processing and analysis system to integrate with other instrumentation. Cartridge 1700 includes a receptacle 1708 for receiving a sample container 1704. A sample is received from a patient, for example, with a swab. The swab is then placed into container 1704. Container 1704 is then positioned within receptacle 1708. Receptacle 1708 retains the container and allows the sample to be processed in the analysis system. In certain approaches, receptacle 1708 couples container 1704 to port 1602 so that the sample can be directed from container 1704 and processed though system 1600. Cartridge 1700 may also include additional features, such as ports 1706, for ease of processing the sample. In certain approaches, ports 1706 correspond to ports of system 1600, such as ports 1602, 1612, 1626, 1634, 1638, and 1650 to open or close to ports or apply pressure for moving the sample through system 1600.

Cartridges may use any appropriate formats, materials, and size scales for sample preparation and sample analysis. In certain approaches, cartridges use microfluidic channels and chambers. In certain approaches, the cartridges use macrofluidic channels and chambers. Cartridges may be single layer devices or multilayer devices. Methods of fabrication include, but are not limited to, photolithography, machining, micromachining, molding, and embossing.

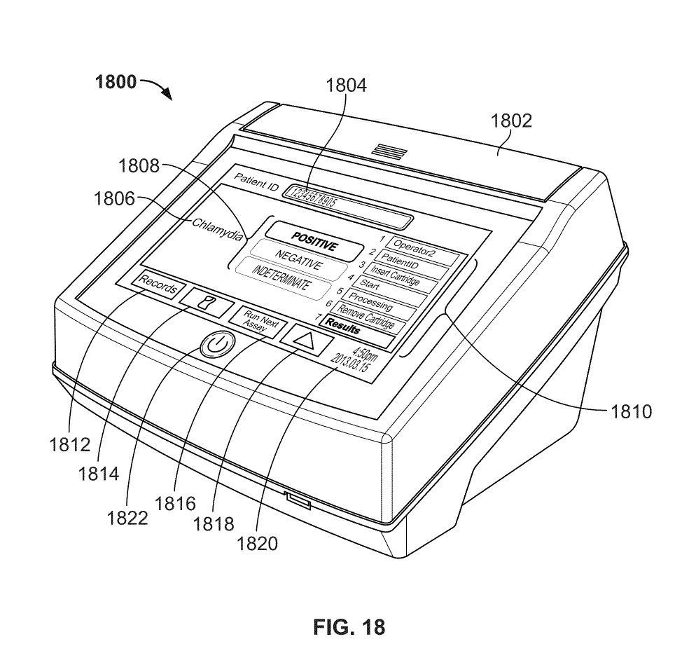

FIG. 18 depicts an automated testing system to provide ease of processing and analyzing a sample. System 1800 may include a cartridge receiver 1802 for receiving a cartridge, such as cartridge 1700. System 1800 may include other buttons, controls, and indicators. For example, indicator 1804 is a patient ID indicator, which may be typed in manually by a user, or read automatically from cartridge 1700 or cartridge container 1704. System 1800 may include a “Records” button 1812 to allow a user to access or record relevant patient record information, “Print” button 1814 to print results, “Run Next Assay” button 1818 to start processing an assay, “Selector” button 1818 to select process steps or otherwise control system 1800, and “Power” button 1822 to turn the system on or off Other buttons and controls may also be provided to assist in using system 1800. System 1800 may include process indicators 1810 to provide instructions or to indicate progress of the sample analysis. System 1800 includes a test type indicator 1806 and results indicator 1808. For example, system 1800 is currently testing for Chlamydia as shown by indicator 1806, and the test has resulted in a positive result, as shown by indicator 1808. System 1800 may include other indicators as appropriate, such as time and date indicator 1820 to improve system functionality.

The foregoing is merely illustrative of the principles of the disclosure, and the systems, devices, and methods can be practiced by other than the described embodiments, which are presented for the purposes of illustration and not of limitation. It is to be understood that the systems, devices, and methods disclosed herein, while shown for use in detection systems for bacteria, and specifically, for Chlamydia Trachomatis, may be applied to systems, devices, and methods to be used in other applications including, but not limited to, detection of other bacteria, viruses, fungi, prions, plant matter, animal matter, protein, RNA sequences, DNA sequences, as well as cancer screening and genetic testing, including screening for genetic disorders.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited are hereby incorporated by reference herein in their entireties and made part of this application.

1-241. (canceled)

242. A method for detecting the presence of a pathogen in a biological host, the method comprising:
providing a sample from the biological host;
providing a biosensor having a first probe configured to detect a control marker in the sample, the control marker being an endogenous element of the biological host, and a second probe configured to detect the presence of a target marker in the sample, the target marker being from a pathogen in the biological host;
applying the sample to the biosensor;
identifying, using the first probe, the presence or absence of the control marker in the sample; and
identifying, using the second probe, the presence or absence of the target marker in the sample.

243. The method of claim 242, wherein the biosensor has a first sensor and a second sensor, and wherein the first probe is coupled to the first sensor and the second probe is coupled to the second sensor.

244. The method of claim 243, further comprising applying an electrochemical lysing procedure to the sample before applying the sample to the biosensor.

245. The method of claim 244, wherein identifying the presence or absence of a marker in the sample comprises measuring an electrocatalytic signal at the biosensor.

246. The method of claim 245, wherein at least one of the control marker and the target marker comprises a ribonucleic acid sequence.

247. The method of claim 246, wherein at least one of the first and second probes comprises a peptide nucleic acid sequence tethered to the biosensor with a thiol bond.

248. The method of claim 242, further comprising:
receiving a first biosensor signal indicative of the presence of the control marker in the sample;
receiving a second biosensor signal indicative of the presence of the target marker in the sample; and
determining, based on the first biosensor signal and the second biosensor signal, that the pathogen is present in the biological host.

249. The method of claim 242, further comprising:
receiving a first biosensor signal indicative of the presence of the control marker in the sample;
receiving a second biosensor signal indicative of the absence of the target marker in the sample; and
determining, based on the first biosensor signal and the second biosensor signal, that the pathogen is not present in the biological host.

250. The method of claim 242, further comprising:
receiving a first biosensor signal indicative of the absence of the control marker in the sample; and
determining an error based on the first signal.

251. The method of claim 242, wherein the biosensor has a third probe, wherein the third probe is a non-sense probe comprising a peptide nucleic acid.

252. A biosensor comprising:
a solid support base;
a sensor affixed to the support base; and
wherein:
the sensor includes a first probe configured to detect the presence of a control marker, the control marker being an endogenous element of a biological host; and
a second probe configured to detect the presence of a target marker, the target marker being from a pathogen in the biological host.

253. The method of claim 252, further comprising performing a baseline measurement using the second probe before applying the sample to the biosensor.

254. The biosensor of claim 253, wherein the first probe comprises a nucleic acid sequence tethered to the sensor, and the second probe comprises a peptide nucleic acid sequence tethered to the sensor.

255. The method of claim 254, wherein identifying the presence of the control marker comprises applying a voltage signal to the first probe, and measuring a current signal from a first electrode.

256. The method of claim 255, wherein identifying the presence of the target marker comprises applying a voltage signal to the second probe, and measuring a current signal from a second electrode.

257. The method of claim 256, further comprising applying an electrocatalytic reagent to the biosensor.

258. The method of claim 252, wherein the first probe and second probe are located in a chamber, and wherein applying the sample to the biosensor comprises flowing the sample through the chamber at a flow rate.

259. The method of claim 258, wherein applying the sample to the biosensor comprises agitating the sample.

260. The method of claim 252, wherein the biosensor has a third sensor having a third probe, wherein the third probe is a non-sense probe.

261. The method of claim 252, wherein the first sensor is a nanostructured microelectrode.
